(19) United States
(12) Patent Application Publication
(10) Pub. No.: US 2008/0226729 A1
(43) Pub. Date: Sep. 18, 2008

(54) STABLE POWDER FORMULATIONS OF ALUM-ADSORBED VACCINES

(75) Inventors: Vincent J. Sullivan, Cary, NC (US); John A. Mikszta, Durham, NC (US); Jason B. Alarcon, Durham, NC (US); Matthew S. Ferriter, Chapel Hill, NC (US); Joanne Huang, Cary, NC (US); Ajit M. D'Souza, Cary, NC (US)

Correspondence Address: David W. Highet, VP & Chief IP Counsel;Becton, Dickinson and Company, (Alston & Bird LLP), 1 Becton Drive, MC 110, Franklin Lakes, NJ, 07417-1880, US

(73) Assignee: Becton, Dickinson and Company, Franklin Lake, NJ (US)

(21) Appl. No.: 11/852,769

(22) Filed: Sep. 10, 2007

Related U.S. Application Data
(60) Provisional application No. 60/843,032, filed on Sep. 8, 2006.
(60) Provisional application No. 60/890,712, filed on Feb. 20, 2007.
(60) Provisional application No. 60/891,628, filed on Feb. 26, 2007.
(60) Provisional application No. 60/918,886, filed on Mar. 19, 2007.

(51) Int. Cl. A61K 9/14; A61K 39/00; A61P 37/00

(52) U.S. Cl. 424/489; 424/236.1; 424/246.1; 424/243.1; 424/227.1

(57) ABSTRACT

The present invention is directed to methods for preparing a stable powder formulation of an alum-adsorbed vaccine. The methods comprise atomizing a liquid formulation comprising an immunogen adsorbed onto an aluminum adjuvant to produce an atomized formulation, freezing the atomized formulation to produce frozen particles, and drying the frozen particles to produce dried powder particles. Pharmaceutical compositions comprising a stable powder formulation of an alum-adsorbed vaccine are also disclosed herein. The pharmaceutical compositions are stable at high temperatures and can be reconstituted in a pharmaceutically acceptable carrier to produce a reconstituted liquid vaccine that exhibits little or no particle agglomeration and retains immunogenicity. Methods of using the alum-adsorbed vaccine compositions for preventing and treating a disease in a subject, wherein the disease is associated with the particular immunogen, are further provided.

### CROSS REFERENCE TO RELATED APPLICATIONS

This application claims the benefit of U.S. Provisional Application No. 60/843,032, filed on Sep. 8, 2006, U.S. Provisional Application No. 60/890,712, filed on Feb. 20, 2007, U.S. Provisional Application No. 60/891,628, filed on Feb. 22, 2007, and U.S. Provisional Application No. 60/918,886, filed on Mar. 19, 2007, all of which are herein incorporated by reference in their entirety.

### FEDERALLY SPONSORED RESEARCH OR DEVELOPMENT

This invention was made with government support under contract number DAMD17-03-2-0037 awarded by the United States Medical Research and Materiel Command. The government has certain rights in the invention.

### FIELD OF THE INVENTION

The present invention relates to methods of preparing stable powder formulations of alum-adsorbed vaccines, pharmaceutical compositions comprising stable powder formulations of dried vaccine particles, and methods of using these compositions in the prevention and treatment of disease.

### BACKGROUND OF THE INVENTION

Hepatitis B is a serious viral liver infection that is transmitted by exposure to infected blood and bodily fluids. An estimated twelve million Americans and two billion people worldwide have been infected with hepatitis B. While most healthy adults infected with hepatitis B will recover and develop protective antibodies, a significant number of patients, particularly infants and children, will develop chronic hepatitis B infections that can lead to life-threatening liver cirrhosis, liver failure, or liver cancer. Approximately one million people worldwide die from hepatitis B infections each year. See, for example, the Hepatitis B Foundation website at hepb.org/index.html.

The hepatitis B virus is a DNA virus that contains an inner core and an outer envelope. The outer envelope of the vaccine comprises a protein referred to as the “hepatitis B surface antigen” or “HBsAg.” The inner core contains the viral DNA and the DNA polymerase enzymes used in viral replication. The inner core antigenic agent is frequently referred to in the art as “HBcAg.” Commercial vaccines for hepatitis B are available, including Engerix-B (GlaxoSmithKline, Inc.) and Recombivax HB (Merck & Co.), but these liquid formulations are optimally stored and transported under refrigerated conditions and are not designed to withstand extreme conditions (e.g., high temperatures, freeze-thaw cycles, long-term storage, etc.). Accordingly, the development of hepatitis B vaccines as stable powder formulations that are suitable for storage and transportation under such extreme conditions would be beneficial.

In addition to a recognition of the advantageous properties of stable powder vaccine formulations for more “traditional” diseases such as hepatitis B, recent world events have raised significant interest in developing similar vaccine formulations that could be use prophylactically or therapeutically to combat the use of biological compounds as bioterrorist agents. Botulinum neurotoxins (BoNTs) are among the most toxic proteins to humans and, therefore, represent a likely biological weapon for use by terrorists. Botulism is a potentially deadly neurological disorder in which BoNT binds to the synapses of motor neurons and prevents the release of the neurotransmitter acetylcholine. As a result, exposure to a BoNT can lead to blurred vision, dysphagia, general respiratory and musculoskeletal paralysis, and death caused by respiratory or cardiac failure within a few days of exposure to the toxin. Seven different serotypes of the bacterium Clostridium botulinum are known, and each strain produces a different form of BoNT, designated BoNT/A, B, C, D, E, F, and G. The most widely studied BoNT, BoNT/A, is synthesized in a specific C. botulinum strain as an approximately 150 kDa single chain protein. This single chain protein is cleaved to produce a 100 kDa heavy chain (HC) and a 50 kDa light chain (LC) linked by a disulfide bond. See, for example, Li and Singh (2000) Biochem. 39:6466-6474 and Swaminathan and Eswaramoorthy (2000) Nature Structural Biol: 7:693-699.

Bacillus anthracis is the causative agent of the pulmonary (i.e., inhalational), cutaneous, and gastrointestinal forms of anthrax. The possibility of creating aerosolized anthrax spores has made B. anthracis a bioterrorist agent of choice. Inhalational anthrax, which would result from an aerosolized or weaponized form of this bacterium, has a fatality rate of nearly 100% if not treated shortly after exposure and prior to the development of symptoms. Patients suffering from inhalational anthrax generally present initially with a high fever and chest pain that rapidly progresses to a systemic hemorrhagic pathology and cardiac or respiratory arrest. Approximately ninety strains of B. anthracis are known, ranging from benign strains to highly virulent strains that could be used as biological weapons. Virulent B. anthracis strains comprise a poly-D-glutamyl capsule, which is itself nontoxic but mediates the invasive stage of the disease, and a multi-component toxin. The anthrax toxin has three distinct antigenic components, each of approximately 80 kDa, designated the edema factor (“EF” or “Factor I”), the protective antigen (“PA” or “Factor II”), and the lethal factor (“LF” or “Factor III”). The protective antigen comprises the binding domain of the anthrax toxin and is so-named because it induces protective antitoxic antibodies when administered to certain mammals. Previous research has established that the lethal factor is necessary to produce the lethal effects exhibited by the anthrax toxin. The combination of only the lethal factor and the protective antigen has been shown to be lethal in experimental animals. See Bravata et al. (2006) Annals Intern. Med. 144(4):270-280; Todor's Online Textbook of Bacteriology: Bacillus anthracis and anthrax at textbookofbacteriology.net/Anthrax.html (University of Wisconsin-Madison Department of Microbiology); and Brock Biology of Microorganisms (M. Madigan and J. Martinko, eds.; Prentis Hall, 2005) for general discussions of B. anthracis and anthrax. Despite the significant threat of a bioterrorist attack with B. anthracis, only one anthrax vaccine is currently used in the U.S. and multiple doses must be administered over a period of months to elicit protective immunity.

Staphylococcal enterotoxin B (SEB) is an approximately 28 kDa enterotoxin produced by the bacterium Staphylococcus aureus and is traditionally associated with food poisoning resulting from unrefrigerated meats and dairy products. Classic signs of food poisoning caused by SEB are an abrupt onset of gastrointestinal symptoms that are generally self-limiting and resolve within twenty-four hours. Of greater concern in the current international political climate is the fact that SEB is a potential bioterrorist agent. SEB is stable, easily aerosolized, and can cause systemic damage, multi-organ system failure, septic shock, and even death when inhaled at very high levels. Symptoms of inhalation of SEB include but are not limited to high fever, shortness of breath, severe chest pain, and, in severe cases, pulmonary edema and adult respiratory distress syndrome (ARDS). Accordingly, SEB, particularly aerosolized SEB, represents a significant bioterrorist threat.

The gram-negative bacterium Yersinia pestis is the causative of the plague. Y. pestis comprises a fraction 1 capsular antigen (i.e., “F1”), which confers anti-phagocytic properties to the bacterial cells, and a V antigen that suppresses the host's innate immune response. A fusion protein comprising the two antigens, designated F1-V, has been produced. See, for example, Santi et al. (2006) Proc. Natl. Acad. Sci. USA 103:861-866.

Three clinical forms of plague exist in humans: bubonic, septicemic, and pneumonic plague. Pneumonic plague is the most serious form of Y. pestis infection and occurs when the bacteria infect the lungs and cause pneumonia. Primary pneumonic plague results from direct inhalation of the Y. pestis bacteria, such as by airborne transmission from an infected person to an uninfected individual or by intentional release of aerosolized bacteria (e.g., a bioterrorist attack). Kool (2005) Healthcare Epidemiology 40:1166-1172. Pneumonic plague has an incubation period of approximately 1-6 days and is characterized by, for example, the sudden onset of severe headache, chills, malaise, and increased respiratory and heart rates. Id. These symptoms rapidly progress to pneumonia and may ultimately lead to respiratory failure and death if left untreated. See Josko (2004) Clin. Lab. Sci. 17:25-29. Appropriate antibiotics, if administered in a timely fashion (i.e., within approximately 20 hours of the onset of the disease) reduce mortality rates, but fatalities resulting from pneumonic plague remain high, particularly in the event of a bioterrorist event in which numerous individuals could be exposed and the national stockpile of antibiotics could be rapidly depleted.

The CDC and Homeland Security have deemed Y. pestis a logical candidate for a potential bioterrorist weapon, one which poses a particularly dangerous threat because of: 1) its natural occurrence on every continent, 2) the ease of its dissemination from wild and domesticated animal reservoirs, as well as man-made devices, 3) a lack of current experience with its clinical presentation coupled possibly with physician complacency in this era of readily available antibiotics, 4) the ability to mass produce the bacteria, 5) the ease by which genetically modified, antibiotic-resistant strains can be produced and aerosolized, and 6) the fact that primary pneumonic plague can be spread from person to person via inhalation of contaminated aerosol droplets. See, for example, Finegold et al. (1968) Am. J. Path. 53:99-114; Walker (1968) Curr. Top. Micro. Immun. 41:23-42; Walker (1968) J. Infect. Dis. 118:188-96; Beebe and Pirsch (1958) Appl. Microbiol. 6:127-138; Williams et al. (1994) J. Wildlife Dis. 30:581-585; Watson et al. (2001) Veter. Path. 38:165-172; and Green et al. (1999) Med. Micro. 23:107-113.

The potential use of a BoNT, a virulent strain of B. anthracis, SEB, or Y. pestis (e.g., F1-V) as bioterrorist agents makes the development of stable powder vaccines against a BoNT, anthrax, SEB, and/or Y. pestis advantageous, particularly if such vaccines could be administered by a variety of techniques, including minimally-invasive methods, and by medical or non-medical personnel. Stabilized BoNT, anthrax, SEB, and Y. pestis vaccine formulations could be readily reconstituted to permit the prophylactic immunization of first responders, military personnel, and possibly even the general population in the event or threat of a bioterrorist attack. Stable polyvalent vaccines that provide protective immunity against a plurality of bioterrorist agents would be particularly advantageous.

Vaccines, such as hepatitis B and anthrax vaccines, typically contain at least one adjuvant to enhance a subject's immune response to the immunogen. Aluminum salts are frequently used as adjuvants to boost the immunogenicity of vaccines. The application of traditional approaches for stabilizing liquid biological products for the storage of alum-adsorbed vaccines, however, has been problematic. In particular, alum-adsorbed vaccines typically exhibit agglomeration, decreased immunogen concentration, and loss of immunogenicity when subjected to conventional lyophilization, freezing, and freeze-drying processes. See, for example, Maa et al. (2003) J. Pharm. Sci. 92:319-332; Diminsky et al. (1999) Vaccine 18:3-17; Alving et al. (1993) Ann. NY Acad. Sci. 690:265-275; and Warren et al. (1986) Ann. Rev. Immunol. 4:369-388, all of which are herein incorporated by reference. The use of conventional methods to produce stable powdered formulations of alum-adsorbed vaccines that can be reconstituted without a loss of stability and immunogenicity has been largely unsuccessful. Therefore, pharmaceutical compositions comprising stable powder forms of alum-adsorbed vaccines that address the problems of agglomeration, loss of immunogenicity, and decreases in immunogen concentration. The resulting stable powder vaccines should be readily reconstitutable in a diluent to produce efficacious liquid vaccines that exhibit little or no particle agglomeration or loss of immunogenicity or immunogen concentration. Such methods would facilitate long-term storage of alum-adsorbed vaccines, extend the shelf-life of these vaccines, permit their use in areas where refrigerated storage and transportation are unavailable, allow for vaccine administration to subjects by a variety of techniques (e.g., potentially minimally invasive administration methods that would not require medical personnel) and, particularly with respect to bioterrorist agents, facilitate stockpiling of the vaccine.

### BRIEF SUMMARY OF THE INVENTION

The present invention is directed to methods for preparing a stable powder formulation of an alum-adsorbed vaccine. The methods comprise atomizing a liquid formulation comprising an immunogen adsorbed onto an aluminum adjuvant to produce an atomized formulation, freezing the atomized formulation to produce frozen particles, and drying the frozen particles to produce dried powder particles. Drying of the frozen particles may be performed at about atmospheric pressure, particularly in the presence of vibration, internals, and/or mechanical stirring.

The pharmaceutical compositions of the invention include vaccines in particulate, powder form that are stable even when subjected to non-optimal conditions (e.g., high temperatures, freeze-thaw, etc.). The powder vaccine formulations disclosed herein can be reconstituted in a diluent to produce reconstituted liquid vaccines that may exhibit little or no particle agglomeration, display no significant decrease in immunogen concentration, retain a substantial level of immunogenicity and/or antigenicity, and maintain protective efficacy against the disease or disorder of interest. Moreover, the powder and reconstituted vaccine formulations of the invention may be suitable for administration by medical or non-medical personnel by a variety of methods, particularly minimally invasive administration techniques. Pharmaceutical compositions comprising stable powder formulations of alum-adsorbed vaccines (or reconstituted liquid forms thereof) and methods of using these compositions for preventing and treating particular diseases, disorders, and the symptoms thereof are also provided.

### BRIEF DESCRIPTION OF THE DRAWINGS

FIG. 1A provides the sedimentation rate curves obtained with various liquid and spray-freeze-dried (SFD) Shanvac-B hepatitis B vaccine formulations at day 0. The sedimentation rates following storage of the vaccines at 4° C. for 28 days are provided in FIG. 1B. Data for the following formulations are presented: Formulation 1 (liquid Shanvac-B vaccine); Control 2 (liquid Shanvac-B vaccine+dextran/trehalose); Formulation 2 (SFD Shanvac-B vaccine+dextran/trehalose); Control 3 (liquid Shanvac-B vaccine+mannitol/trehalose); and Formulation 3 (SFD Shanvac-B vaccine+mannitol/trehalose). Experimental details are provided in Example 2.

FIG. 2A provides the sedimentation rate curves obtained with the liquid Shanvac-B hepatitis B vaccine stored at 55° C. for 0, 7, 14, and 28 days. FIG. 2B provides the sedimentation rates of SFD Shanvac-B vaccine+mannitol/trehalose stored at 55° C. for 0, 7, 14, and 28 days. Sedimentation data obtained with a control sample (i.e., liquid Shanvac-B vaccine+mannitol/trehalose) are also presented in FIG. 2B for purposes of comparison. Experimental details are provided in Example 2.

FIG. 3A provides the sedimentation rate curves obtained with the liquid Shanvac-B hepatitis B vaccine following a freeze-thaw cycle and storage at 55° C. for 0 and 14 days. FIG. 3B provides the sedimentation rates of SFD Shanvac-B vaccine+mannitol/trehalose following a freeze-thaw cycle and storage at 55° C. for 0 and 14 days. Experimental details are provided in Example 2.

FIG. 4 provides the mean anti-HBsAg antibody concentration (μg/ml) in serum from mice immunized with the specified liquid or SFD vaccine formulations at 28 days (FIG. 4A) and 42 days (FIG. 4B) post-immunization. Mice were immunized at day 0 and day 28. Data for the following formulations are presented: Liquid Shanvac-B vaccine; liquid Shanvac-B vaccine+dextran/trehalose; liquid Shanvac-B vaccine+mannitol/trehalose; SFD Shanvac-B vaccine+dextran/trehalose; SFD Shanvac-B vaccine+mannitol/trehalose; and Engerix-B (control hepatitis B vaccine; manufactured by Merck & Co.). Experimental details are provided in Example 2.

FIG. 5 provides the sedimentation rate curves obtained with the liquid (FIG. 5A) and atmospheric spray-freeze-dried (ASFD; FIG. 5B) Shanvac-B hepatitis B vaccine formulations following storage at 4° C. for 0 and 28 days. Experimental details are provided in Example 3.

FIG. 6 provides the sedimentation rate curves obtained with the liquid (FIG. 6A) and ASFD (FIG. 6B) Shanvac-B hepatitis B vaccine formulations following storage at 55° C. for 0, 7, 14, and 28 days. Experimental details are provided in Example 3.

FIG. 7 provides the sedimentation rate curves obtained with the liquid and ASFD Shanvac-B vaccine formulations following a freeze-thaw cycle and storage at 55° C. for 14 days. Experimental details are provided in Example 3.

FIG. 8 provides the mean anti-HBsAg antibody concentration (μg/ml) in serum from mice immunized with the specified liquid or ASFD vaccine formulations at 28 and 42 days post-immunization. Mice were immunized at day 0 and day 28. Data for the following formulations are presented: Liquid Shanvac-B vaccine; liquid Shanvac-B vaccine+mannitol/trehalose; ASFD Shanvac-B vaccine+mannitol/trehalose; and SFD Shanvac-B vaccine+mannitol/trehalose. Experimental details are provided in Example 3.

FIG. 9 provides the mean serum antibody titers following immunization with 1.0 μg or 0.1 μg of a liquid or reconstituted SFD powder Botulinum neurotoxin A (BoNT/A) vaccine. The vaccine formulations were administered by intramuscular (IM), intradermal (ID), or intranasal (IN) administration, as indicated in the Figure, on days 0 and 28. Mean serum antibody titers are provided for days 14, 28 and 42 (FIGS. 9A, B, and C, respectively). The number of mice from the various test groups surviving at day 54 following lethal challenge with BoNT/A at day 49 are also set forth in FIG. 9C. The dashed line of FIG. 9C indicates an antibody titer of 800, at which level subjects exhibit a 97% survival rate from lethal challenge with BoNT/A. Further experimental details are set forth in Example 4.

FIG. 10 provides the mean serum antibody titers following immunization with 1.0 μg or 0.1 μg of a liquid or reconstituted SFD powder BoNT vaccine. The vaccine formulations were administered by intramuscular (IM), intradermal (ID), or intranasal (IN) administration, as indicated in the Figure, on days 0 and 28. Mean serum antibody titers are provided for days 14, 28 and 42 (FIGS. 10A, B, and C, respectively). The number of mice from the various test groups surviving at day 54 following lethal challenge with BoNT/A at day 49 are also set forth in FIG. 10C. The dashed line of FIG. 10C indicates an antibody titer of 800, at which level subjects exhibit a 97% survival rate from lethal challenge with BoNT. Additional experimental details are provided in Example 4.

FIG. 11 provides the neutralizing antibody titers in serum following immunization with various B. anthracis rPA vaccine formulations, as determined by the anthrax lethal toxin neutralization assay. The specific details of the vaccine formulations used with each animal test group are set forth in Table 20. Additional experimental details are presented in Example 5.

FIG. 12 provides the endpoint neutralizing antibody titers in serum following immunization with various B. anthracis rPA vaccine formulations, as determined by the anthrax lethal toxin neutralization assay. The specific details of the vaccine formulations used with each animal test group are set forth in Table 21. Additional experimental details are presented in Example 5.

FIG. 13 provides the mean serum antibody titers following immunization with 10 μg or 3.3 μg of a liquid or reconstituted SFD powder F1-V vaccine. The vaccine formulations were administered intramuscularly to test mice on days 0 and 28. Mean serum antibody titers are provided for days 14, 28 and 42 (FIGS. 13A, B, and C, respectively). Circles in these figures represent serum antibody titers for individual mice, and the bars provide the mean antibody titer for all of the test animals. Additional experimental details are provided in Example 6.

FIG. 14 provides the geometric mean serum antibody titers from mice immunized with either polyvalent liquid vaccine pre-adsorbed to aluminum hydroxide adjuvant (“Liquid Poly”), reconstituted SFD powder vaccine pre-adsorbed to aluminum hydroxide (“SFD Poly, Pre-adsorbed”), or monovalent liquid vaccine control (“Liquid Mono”). Serum samples were screened for antibodies against constituent antigens of the polyvalent vaccine, i.e. recombinant Protective Antigen (rPA) from Bacillus anthracis (FIG. 14A), Staphyloccocal Enterotoxin B (SEB) from Staphlycoccus aureus (FIG. 14B), Botulinum Neurotoxin (BoNT) from Clostridium botulinum (FIG. 14C), and F1-V fusion protein from Yersinia pestis (FIG. 14D). Mice were immunized at days 0 and 28, as indicated by arrows, and blood was obtained for antibody analysis at days 0, 28 and 42. Additional experimental details are provided in Example 7.

### DETAILED DESCRIPTION OF THE INVENTION

The present invention is directed to methods for preparing a stable powder formulation of an alum-adsorbed vaccine, such as a hepatitis B, Botulinum neurotoxin (BoNT), anthrax (i.e., B. anthracis), plague (i.e., Y. pestis), or Staphylococcal enterotoxin (i.e., Staphylococcal enterotoxin B (SEB) from S. aureus) vaccine. The methods for producing stable alum-adsorbed vaccine powder formulations disclosed herein generally comprise spray-freeze-drying (SFD) or atmospheric spray-freeze-drying (ASFD) techniques, such as those described in U.S. Patent Application Publication No. 2003/0180755 and Jiang et al. (2006) J. Pharm. Sci. 95:80-96, both of which are incorporated by reference in their entirety. The pharmaceutical vaccine powder compositions and methods of using these compositions are also encompassed by the present invention.

As used herein, the term “alum-adsorbed vaccine” refers to an immunogenic composition that comprises an immunogen and an aluminum adjuvant, particularly wherein the immunogen is adsorbed onto the aluminum adjuvant. Aluminum adjuvants are well known in the art and include, for example, aluminum salts such as aluminum hydroxide, aluminum phosphate, and aluminum sulfate. The term “alum” encompasses any aluminum adjuvant. In particular embodiments, the aluminum adjuvant is aluminum hydroxide (e.g., Alhydrogel®).

The disclosed alum-adsorbed vaccine powder formulations are stable when stored at high temperatures (i.e., above conventional refrigeration temperatures) and/or subjected to freeze-thaw. The alum-adsorbed vaccine powder formulations can be readily reconstituted in a diluent to produce a reconstituted liquid vaccine that exhibits little or no particle agglomeration, displays no significant decrease in immunogen concentration, retains a substantial level of immunogenicity and/or antigenicity, and exhibits a significant level of protection against the disease-causing pathogen or toxin of interest (i.e., “protective efficacy” or “protective immunity”). Methods for preparing reconstituted liquid alum-adsorbed vaccines are also disclosed. Methods for using the alum-adsorbed vaccine powder compositions (or reconstituted liquid formulations thereof) in the prevention or treatment of particular diseases, disorders, or symptoms associated with exposure to a particular disease-causing pathogen or toxin are further provided. Furthermore, the readily reconstitutable nature of the stable powder vaccine formulations disclosed herein may permit administration of the reconstituted vaccines by a variety of methods. In certain aspects of the invention, a reconstituted vaccine may be administered by minimally invasive techniques, with or without the assistance of medically trained personnel. For example, a reconstituted vaccine of the invention, particularly a hepatitis B, anthrax, BoNT vaccine, more particularly a BoNT/A vaccine, a plague vaccine, more particularly an F1-V plague vaccine, or a Staphylococcal enterotoxin vaccine, more particularly an SEB vaccine, may be administered intradermally by a microneedle.

Methods for preparing a stable powder formulation of an alum-adsorbed vaccine comprise atomizing a liquid formulation that comprises an immunogen adsorbed onto an aluminum adjuvant to produce an atomized formulation, freezing the atomized formulation to produce frozen particles, and drying the frozen particles to produce dried powder particles. Such methods may be referred to herein as “spray-freeze-drying (SFD).” See, for example, Jiang et al. (2006) J. Pharm. Sci. 95:80-96; Maa et al. (2003) J. Pharm. Sci. 92:319-332; and U.S. Patent Application Publication No. 2003/0180755, all of which are herein incorporated by reference. In a particular aspect of the invention, the claimed methods for preparing a stable powder formulation of an alum-adsorbed vaccine comprise atomizing a liquid formulation comprising an immunogen adsorbed onto an aluminum adjuvant to produce an atomized formulation, freezing the atomized formulation to produce frozen particles, and drying the frozen particles at about atmospheric pressure to produce dried powder particles. The drying step may be performed in the presence of vibration, internals, mechanical stirring, or a combination thereof. This method of producing powder formulations may be referred to as “atmospheric spray-freeze-drying (ASFD).” See U.S. Patent Application Publication No. 2003/0180755.

Conventional liquid formulations of alum-adsorbed vaccines have been shown to lose immunogenicity and to aggregate when subjected to traditional lyophilization, freezing, and freeze-drying techniques that are used to facilitate long-term storage. See, for example, Maa et al. (2003) J. Pharm. Sci. 92:319-332; Diminsky et al. (1999) Vaccine 18:3-17; Alving et al. (1993) Ann. NY Acad. Sci. 690:265-275; and Warren et al. (1986) Ann. Rev. Immunol. 4:369-388, all of which are herein incorporated by reference. As a result, alum-adsorbed vaccines must generally be stored and transported as liquid formulations under refrigerated conditions (e.g., at about 2° C. to about 8° C.). The methods of the present invention, however, permit the production of a stable powder formulation of an alum-adsorbed vaccine, such as a hepatitis B, BoNT, anthrax, plague (e.g., F1-V), or Staphylococcal enterotoxin (e.g., SEB) vaccine, that can be stored under non-optimal conditions (e.g., non-refrigerated conditions) and that can be reconstituted in a suitable carrier to produce a reconstituted liquid vaccine that exhibits little or no particle agglomeration, retains immunogenicity/immunogenicity, and maintains protective efficacy against the disease, toxin, or symptoms associated therewith. “Non-optimal conditions” or “non-optimal storage conditions” as used herein generally refer to conditions such as storing the vaccine composition at high temperatures, subjecting the vaccine formulation to one or more freeze-thaw cycles, and storing the vaccine composition for prolonged time periods. By “high temperature” is intended temperatures above the refrigeration conditions traditionally recommended for storage of liquid vaccine formulations and may include, for example, temperatures of 10°, 20°, 30°, 40°, 50°, 55° C. or higher.

By “powder” or “powder formulation” or “pharmaceutical composition comprising a powder formulation” is intended a composition that consists of substantially solid, free-flowing particles. A “stable powder formulation” or a “pharmaceutical composition comprising a stable powder formulation of an alum-adsorbed vaccine” of the invention maintains substantial structural integrity (e.g., displays little or no agglomeration, maintains a substantial amount of the original immunogen concentration, etc.) and retains a substantial level of immunogenicity, antigenicity, and/or protective efficacy relative to that of the original liquid formulation. In particular aspects of the invention, a powder formulation of an alum-adsorbed vaccine is stable even when subjected to storage under non-optimal conditions (e.g., high temperatures, freeze-thaw cycles, long-term storage, etc.). For example, a stable powder formulation of the invention may be stored under non-optimal conditions and reconstituted to produce a liquid vaccine formulation, wherein the reconstituted liquid vaccine exhibits little or no particle agglomeration, maintains a substantial amount of the original immunogen concentration, and further retains a substantial level of immunogenicity and/or antigenicity, as described further herein below. Stability of an alum-adsorbed vaccine composition may be assessed by measuring, for example, the rate of sedimentation, which corresponds to the extent of particle agglomeration, and the concentration of immunogen present in the reconstituted liquid vaccine. In particular, the rate of sedimentation and concentration of immunogen of the reconstituted liquid vaccine may be compared with that of the original liquid formulation (i.e., the liquid formulation comprising the immunogen adsorbed onto an aluminum adjuvant prior to atomization). Standard assays for measuring the rate of sedimentation, concentration of immunogen, immunogenicity, and antigenicity are known in the art and described in Examples 2 and 3. Protective efficacy may be assessed by, for example, evaluating the survival rates of immunized and non-immunized subjects following challenge with a disease-causing pathogen or toxin associated with a particular immunogen of interest. With regard to the anthrax vaccines of the invention, protective immunity may be analyzed, for example, via anthrax lethal toxin neutralization assays.

The liquid formulations that are atomized in accordance with the methods of the invention include at least one immunogen that is adsorbed onto an aluminum adjuvant. An “immunogen” is any naturally occurring or synthetic substance that induces an immune response in a subject. A liquid formulation comprising more than one immunogen may be used in the practice of the invention and are referred to generally as a “polyvalent” or “multivalent” alum-adsorbed vaccine. As used herein, the term “immunogenicity” refers to the ability of a substance to induce an immune response when administered to a subject (e.g., a cellular immunogen-specific immune response or a humoral antibody response). The immunogens of the invention may be associated with or derived from any pathogen of interest and include, for example, whole cells, viral particles (e.g., partially or completely inactivated viruses), polypeptides, polynucleotides, carbohydrates, lipids, lipoproteins, glycoproteins, and polysaccharides. In particular aspects of the invention, the immunogen comprises a hepatitis B antigen, such as the surface (HBsAg) or core hepatitis B antigen (HBcAg). In other embodiments, the immunogen comprises a botulism immunogen including, for example, a Botulinum neurotoxin such as BoNT/A, B, C, D, E, F, or G. The BoNT immunogen of the invention is typically a BoNT/A antigen, more particularly the BoNT/A heavy chain (HC). The immunogens of the invention also include B. anthracis antigens, particularly B. anthracis toxins or components thereof, more particularly the B. anthracis protective antigen (PA). In certain embodiments, the immunogen is a recombinant B. anthracis Protective Antigen (rPA). Y. pestis antigens of the invention include, for example, the F1 antigen, the V antigen, and, particularly, the F1-V fusion protein antigen. Immunogens further include antigens of Staphylococcal enterotoxins, such as SEB, particularly recombinant SEB (rSEB). In some aspects of the invention, multiple immunogens may be used to produce a polyvalent or multivalent vaccine. Such polyvalent vaccines may comprise, for example, an anthrax antigen (e.g., rPA), a Staphylococcal enterotoxin antigen (e.g., rSEB), a BoNT antigen (e.g., BoNT/A), and a Y. pestis antigen (e.g., rF1-V). Recombinantly produced immunogens and variants or fragments of an immunogen of interest, as defined herein below, may be used to practice the present invention.

Any suitable immunogen as defined herein may be employed. The immunogen may be a viral immunogen. The immunogen may therefore be derived from members of the families Picornaviridae (e.g. polioviruses, etc.); Caliciviridae; Togaviridae (e.g., rubella virus, dengue virus, etc.); Flaviviridae; Coronaviridae; Reoviridae; Birnaviridae; Rhabodoviridae (e.g. rabies virus, etc.); Filoviridae; Paramyxoviridae (e.g. mumps virus, measles virus, respiratory syncytial virus, etc.); Orthomyxoviridae (e.g. influenza virus types A, B and C, etc.); Bunyaviridae; Arenaviridae; Retroviradae (e.g. HTLV-I; HTLV-II; HIV-1 and HIV-2); and simian immunodeficiency virus (SIV) among others.

Alternatively, viral immunogens may be derived from papillomavirus (e.g. HPV); a herpesvirus; a hepatitis virus, e.g. hepatitis A virus (HAV), hepatitis B virus (HBV), hepatitis C(HCV), the delta hepatitis virus (HDV), hepatitis E virus (HEV) or hepatitis G virus (HGV); and the tick-borne encephalitis viruses. See, e.g., Virology 3rd Edition (W. K. Joklik ed. 1988) and Fundamental Virology, 2nd Edition (B. N. Fields and D. M. Knipe, eds. 1991) for a description of these viruses.

Bacterial immunogens for use in the invention can be derived from organisms that cause anthrax, botulism, plague, diphtheria, cholera, tuberculosis, tetanus, pertussis, meningitis and other pathogenic states, including, e.g., Meningococcus A, B and C, Haemophilus influenza type B (HIB), Helicobacter pylori, Vibrio cholerae, Escherichia coli, Campylobacter, Shigella, Salmonella, Streptococcus sp., Staphylococcus sp, Clostridium botulinum, Bacillus anthracis, and Yersinia pestis. A combination of bacterial immunogens may be provided in a single composition comprising, for example, diphtheria, pertussis and tetanus immunogens. Suitable pertussis immunogens are pertussis toxin and/or filamentous haemagglutinin and/or pertactin, alternatively termed P69. An anti-parasitic immunogen may be derived from organisms causing malaria and Lyme disease. In certain aspects of the invention, the bacterial immunogen is selected from the group consisting of recombinant Staphylococcus enterotoxin B (rSEB), Bacillus anthracis recombinant Protective Antigen (rPA), recombinant Clostridium botulinum neurotoxin, and Yersinia pestis F1-V fusion protein. In particular embodiments, combinations of the above immunogens are utilized in the practice of the invention to produce multivalent vaccines.

Immunogens for use in the present invention can be produced using a variety of methods known to those of skill in the art. In particular, the immunogens can be isolated directly from native sources, using standard purification techniques. Alternatively, whole killed, attenuated or inactivated bacteria, viruses, parasites or other microbes may be employed. Yet further, immunogens can be produced recombinantly using known techniques.

Immunogens for use herein may also be synthesized, based on described amino acid sequences, via chemical polymer syntheses such as solid phase peptide synthesis. Such methods are known to those of skill in the art. See, e.g. J. M. Stewart and J. D. Young, Solid Phase Peptide Synthesis, 2nd Ed. (Pierce Chemical Co., Rockford, Ill., (1984)) and G. Barany and R. B. Merrifield, The Peptides. Analysis, Synthesis, Biology, Vol. 2 (E. Gross and J. Meienhofer, eds., Academic Press, New York (1980)), for solid phase peptide synthesis techniques; and M. Bodansky, Principles of Peptide Synthesis, (Springer-Verlag Berlin (1984)) and The Peptides. Analysis, Synthesis, Biology, Vol. 1 E. Gross and J. Meienhofer, eds.) for classical solution synthesis.

In certain aspects of the invention, the liquid formulation comprising an immunogen adsorbed onto an aluminum adjuvant may be a commercially available alum-adsorbed vaccine that is to be formulated as a stable dry powder. A liquid formulation of any alum-adsorbed vaccine may be used to practice the invention. Such vaccines include but are not limited to Infanrix (diphtheria, tetanus, and pertussis), Havrix (pediatric hepatitis A), Vaqta (pediatric hepatitis A), Engerix B (hepatitis B), PedVaxHib (Haemophilus influenza type B), Twinrix (hepatitis A/hepatitis B), Pediarix (diphtheria, tetanus, and pertussis-poliovirus-hepatitis B), Prevnar (pneumococcal conjugate), Daptacel (diphtheria, tetanus, and pertussis), Tripedia (diphtheria, tetanus, and pertussis), Comvax (Haemophilus influenza type B-hepatitis B), Recombivax HB (hepatitis B), Tetrammune (diphtheria, tetanus, and pertussis-Haemophilus influenza type B), Certiva (diphtheria, tetanus, and pertussis), and Shanvac-B (hepatitis B). In particular embodiments, the liquid formulation comprises a hepatitis B alum-adsorbed vaccine, more specifically a vaccine comprising the HBsAg antigen. Moreover, a pentavalent botulinum toxoid vaccine that is adsorbed to aluminum phosphate and that is specific for BoNT/A, B, C, D, and E has been produced for the Centers for Disease Control as an Investigational New Drug. This alum-adsorbed BoNT vaccine may also be used in the practice of the present invention. An anthrax vaccine comprising the protective antigen from an avirulent, non-encapsulated strain of B. anthracis is available in the U.S. but is typically only administered to limited populations (e.g., military personnel, individuals researching B. anthracis, etc.). This liquid anthrax vaccine could be used in the present methods and compositions.

Use of the term a “polynucleotide” or “polynucleotide sequence” is not intended to limit the present invention to polynucleotides comprising DNA. One of skill in the art will appreciate that polynucleotide molecules can comprise ribonucleotides, deoxyribonucleotides, and combinations thereof. A “DNA” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). The terms “polynucleotide” and “nucleic acid” may be used interchangeably herein.

The terms “polypeptide,” “peptide,” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

The polypeptides and polynucleotides used in the practice of the invention can be naturally occurring or recombinantly produced in accordance with routine molecular biology techniques. Variants and fragments of immunogens comprising polypeptides (e.g., HBsAg, BoNT/A, B. anthracis rPA, rSEB, or rF1-V) or polynucleotides are also encompassed by the present invention. “Variants” refer to substantially similar sequences. A variant of an amino acid or nucleotide sequence of the invention will typically have at least about 65%, 70%, 75%, 80%, 85%, 90%, 91%, 92%, 93%, 94%, 95%, 96%, 97%, 98%, 99% or more sequence identity with the reference sequence. In particular embodiments, a variant of an immunogenic polypeptide of the invention will retain the biological activity of the full-length polypeptide and hence be immunogenic. Methods for generating variant sequences are well known in the art as are methods for determining percent identity of polypeptide or polynucleotide sequences, e.g. BLAST.

The term “fragment” refers to a portion of a polypeptide or polynucleotide comprising a specified number of contiguous amino acid or nucleotide residues. In particular embodiments, a fragment of an immunogenic polypeptide of the invention may retain the biological activity of the full-length polypeptide and hence be immunogenic. Fragments of a polynucleotide may encode protein fragments that retain the biological activity of the protein and hence be immunogenic. Fragments of the polypeptides and polynucleotides of the invention can be of any length provided they have the desired attributes (e.g., immunogenicity). Methods for generating fragments of a polypeptide or a polynucleotide are known in the art.

The liquid formulations of the invention comprise an immunogen and an aluminum adjuvant. In addition to the aluminum adjuvant, other adjuvant agents may be used in the practice of the invention. The term “adjuvant” refers to a compound or mixture that enhances the immune response to an immunogen. An adjuvant can serve as a tissue depot that slowly releases the immunogen and also as a lymphoid system activator that non-specifically enhances the immune response (Hood et al., Immunology, Second Ed., 1984, Benjamin/Cummings: Menlo Park, Calif.). Generally, the adjuvants used in the practice of the invention are pharmaceutically acceptable. Such pharmaceutically acceptable adjuvants are well known in the art.

Exemplary adjuvants include, but are not limited to, complete Freund's adjuvant, incomplete Freund's adjuvant, saponin (and derivatives thereof), mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Other adjuvants of interest include CpG DNA, GM-CSF, IL-4, IL-7, IL-12, monophosphoryl lipid A (MPL), 3-Q-desacyl-4′-monophosphoryl lipid A (3D-MLA), IL-1beta 163-171 peptide (Sclavo Peptide), 25-dihydroxyvitamin D3, calcitonin-gene regulated peptides, dehydroepiandrosterone (DHEA), N-Acetylglucosaminyl-(Pl-4)-N-acetylmuramyl-L-alanyl-D-glutamine (GMDP), dimethyl dioctadecyla or disteary ammonium bromide (DDA), Zinc L-proline, formylated-Met-Leu-Phe (fMLP), N-acetyl muramyl-L-threonyl-D-isoglutamine (Threonyl-MDP), N-acetyl-L-alanyl-D-isoglutaminyl-L-alanine-2-(1,2-dipalmitoyl-sn-glycero-3-(hydroxy-phosphoryloxy)ethyl amide monosodium salt (MTP-PE), Nac-Mur-L-Ala-D-Gln-OCH3, Nac-Mur-L-Thr-D-isoGln-sn-glycerol dipalmitoyl, Nac-Mur-D-Ala-D-isoGln-sn-glycerol dipalmitoyl, 1-(2-methypropyl)-1H-imidazo[4,5-c]quinolin-4-amine, 4-Amino-otec-dimethyl-2-ethoxymethyl-1H-imidazo[4,5-c]quinoline-1-ethanol, N-acetylglucosaminyl-N-acetylmuramyl-L-Ala-D-isoGlu-L-Ala-glycerol dipalmitate (DTP-GDP), N-acetylglucosaminyl-N-acetylinuramyl-L-Ala-D-isoGlu-L-Ala-dipalmitoxy propylamide (DTP-DPP), 7-allyl-8-oxoguanosine, poly-adenylic acid-poly-uridylic acid complex, MIP-1 a, MIP-3 a, dibutyl phthalate, dibutyl phthalate analogues and C5a.

The liquid formulations comprising an immunogen adsorbed onto an aluminum adjuvant used to practice the invention may be in any form suitable for atomization, including, for example, a solution, suspension, slurry, or colloid. The liquid formulations may further comprise one or more pharmaceutically acceptable excipients, protectants, solvents, salts, surfactants, and buffering agents. Such excipients are known in the art and may help stabilize the alum-adsorbed vaccines of the invention. Suitable excipients will be compatible with the immunogen and with the aluminum adjuvant and include, for example, water, saline, carbohydrates, glycerol, ethanol, or the like and combinations thereof. Carbohydrate excipients of particular interest include trehalose, mannitol, dextran, cyclodextrin, inulin USP, and combinations thereof. In certain embodiments, the liquid formulations comprise an immunogen, an aluminum adjuvant, and dextran/trehalose or mannitol/trehalose. Furthermore, if desired, the liquid formulation may contain auxiliary substances such as wetting or emulsifying agents and pH buffering agents.

Suitable excipients can include free-flowing particulate solids that do not thicken or polymerize upon contact with water, which are innocuous when administered to an individual, and do not significantly interact with the pharmaceutical agent in a manner that alters its pharmaceutical activity. Examples of normally employed excipients include, but are not limited to, proteins such as human and bovine serum albumin, gelatin, or immunoglobulins, monosaccharides such as glucose, xylose, galactose, fructose, D-mannose or sorbose, disaccharides such as lactose, maltose, saccharose, trehalose or sucrose, sugar alcohols such as mannitol, trehalose, sorbitol, xylitol, glycerol, erythritol or arabitol, polymers such as dextran, starch, cellulose or high molecular weight polyethylene glycols (PEG), amino acids or their salts, such as glycine, alanine, glutamine, arginine, lysine or histidine or their salts with alkali or alkaline earth metals such as a sodium, potassium or magnesium salts, or sodium or calcium phosphates, calcium carbonate, calcium sulfite, sodium citrate, citric acid, tartaric acid, pluronics, surfactants, and combinations thereof. Suitable solvents include, but are not limited to, methylene chloride, acetone, methanol, ethanol, isopropanol and water. Pharmaceutically acceptable salts include, for example, mineral acid salts such as hydrochlorides, hydrobromides, phosphates, sulfates, and the like; and the salts of organic acids such as acetates, propionates, malonates, benzoates, and the like. Chitosan, dermatan sulfate, chondroitin, pectin, and other mucoadhesives may also be used in the practice of the invention, particularly when the vaccine powder formulations are intended for administration by inhalation. Suitable surfactants include but are not limited to Tween 80, pluronics, and the like. A thorough discussion of pharmaceutically acceptable excipients and auxiliary substances is available in Remington's Pharmaceutical Sciences (18^{th} ed.; Mack Publishing Company, Eaton, Pa., 1990), which is incorporated herein by reference.

The methods of the invention for preparing a stable powder formulation of an alum-adsorbed vaccine comprise the steps of atomizing, freezing, and drying. In accordance with the methods of the invention, the steps of atomizing, freezing, and drying may be performed in a single chamber or apparatus, thereby eliminating the possibility of sample contamination and loss of yield. For example, a liquid formulation may be atomized (i.e., sprayed) into a chamber, wherein the freezing of the atomized formulation and the drying of the frozen particles also occur. Exemplary apparatus for atomizing, freezing, and drying in a single chamber are provided in U.S. Patent Application Publication No. 2003/0180755.

The liquid formulations comprising an immunogen adsorbed onto aluminum adjuvant can be atomized using a variety of methods and devices known in the art. For example, the liquid formulation can be sprayed through a two-fluid nozzle, a pressure nozzle, or a spinning disc nozzle or atomized with an ultrasonic nebulizer, an ink jet printer type nozzle, or a vibrating orifice aerosol generator (VOAG). In some aspects of the invention, the liquid formulation is atomized with a pressure nozzle, such as the BD Accuspray® nozzle. Atomization conditions, including atomization gas flow and gas pressure, liquid flow rate, and nozzle size and type, can be varied, particularly to optimize the size of droplets in the atomized formulation and particle size of the resulting dry powder formulation.

Following atomization of the liquid formulation, the droplets are rapidly frozen to produce solid, frozen particles. In particular embodiments, the droplets are frozen immediately after the atomization step. The droplets may be frozen by introducing the atomized formulation into any cold medium having a temperature below the freezing point of the liquid formulation. As used herein, “introducing the atomized formulation into a cold medium” includes any method for contacting the droplets of the atomized formulation with the cold medium, including but not limited to immersing the droplets in a cold liquid or passing the droplets through a cold gas. The term “cold medium” is broadly defined to include any suitable cold liquid or gas that has a temperature below the freezing point of the liquid formulation. Exemplary cold liquids are known in the art and include liquid nitrogen, argon, and hydrofluoroethers. Compressed liquids, such as compressed fluid carbon dioxide, helium, propane, ethane, or equivalent inert liquids, may also be used in the practice of the present invention. The temperature of the cold liquids used during the freezing step are typically between about −200° C. to about −80° C., particularly about −200° C. to about −100° C., more particularly about −200° C. Representative gases for use in the freezing step include but are not limited to cold air, nitrogen, helium, and argon and are generally used at a temperature from between about −5° C. to about −60° C., more particularly about −20° C. to about −40° C. Conventional procedures for obtaining the desired temperature of the cold medium are known in the art. In one embodiment, a liquid formulation comprising the immunogen and the aluminum adjuvant is atomized through a spray nozzle that is positioned above a vessel (e.g., a metal pan) containing liquid nitrogen. The droplets in the atomized formulation generally freeze immediately upon contact with the cold liquid and are collected and dried.

The solid, frozen particles produced during the freezing step of the claimed methods are dried to produce powder particles of the alum-adsorbed vaccine. The term “drying” is used herein to refer to the removal of liquid from the frozen particles to produce powder particles having a moisture content of generally less than 20%, 15%, 10%, 5%, or 1% by weight water.

In some embodiments, such as those involving the spray-freeze-drying (SFD) techniques described above and known in the art, the frozen particles are dried by lyophilization (under vacuum) in accordance with methods and devices known in the art. For example, frozen particles may be collected and transferred to a lyophilizer and the excess liquid evaporated off to yield dried powder particles, as described in Example 1. SFD methods and apparatus are described in, for example, Jiang et al. (2006) J. Pharm. Sci. 95:80-96; Maa et al. (2003) J. Pharm. Sci. 92:319-332; and U.S. Patent Application Publication No. 2003/0180755.

In other aspects of the invention, particularly those involving atmospheric spray-freeze-drying (ASFD) described above, the frozen particles are dried by sublimation in a stream of cold, desiccated gas (e.g., air, nitrogen, or helium) at about atmospheric pressure. As used herein, “about atmospheric pressure” is intended to mean a pressure of approximately 0.5 to five atmospheres, particularly one to three atmospheres, more particularly about one atmosphere of pressure. Methods and apparatus for drying particles at atmospheric pressure are described in U.S. Patent Application No. 2003/0180755 and U.S. Pat. No. 4,608,764.

In a particular embodiment, frozen particles are dried in a cold gas at about atmospheric pressure under conditions that promote fluidization of the particles. Particle fluidization during the drying process prevents channeling and agglomeration and permits faster and more complete particle drying. Any method for enhancing the fluidization of the particles during the drying step may be employed in the practice of the invention. For example, the drying step may be performed in the presence of vibration, internals, mechanical stirring, or a combination thereof. The term “internals” is commonly used in the field of industrial process chemistry and is used herein to refer to any physical barrier (e.g., blades, plates, paddles, or other barriers) positioned inside an apparatus or chamber for SFD or ASFD, wherein the physical barrier is used to promote fluidization of particles during the drying process. See U.S. Patent Application No. 2003/0180755.

In certain aspects of the invention, the frozen particles are dried by a combination of processes, such as sublimation in a cold, desiccated gas stream at about atmospheric pressure followed by conventional lyophilization. For example, the frozen particles may be partially dried by contact with the cold gas, collected on a filter or other collection device, and then subjected to lyophilization to further dry the particles. In accordance with the methods of the invention, the drying process may occur, for example, after deposition and collection of the frozen particles or, alternatively, freezing and drying may occur essentially simultaneously. Any method and container or device for collection of frozen, dried, or partially dried powder particles may be used in the invention. In one embodiment, the dried particles may be collected on a filter from which the particles can be removed for further use or in a pan, as in the case of drying by lyophilization. Once collected, the dried powder particles may be transferred to a sterile container suitable for storage of compositions for use in a medical application.

The dried powder particles of the claimed pharmaceutical compositions may be characterized on the basis of a number of parameters, including but not limited to average particle size (also referred to as average geometric particle size or volume mean diameter), range of particle sizes, mean aerodynamic diameter (also referred to as volume mean aerodynamic diameter), particle surface area, and particle morphology (e.g., particle aerodynamic shape and particle surface characteristics). Methods for assessing these parameters are well known in the art. For example, particle size can be assessed by conventional techniques including but not limited to scanning electron microscopy and laser diffraction. The average particle size of the powder can also be measured as a mass mean aerodynamic diameter (MMAD) using conventional techniques such as cascade impaction. Aerodynamic diameter is defined as the product of the actual particle diameter and the square root of the particle's absolute density, as defined herein below and in the art. If desired, automatic particle-size counters can be used (e.g. Aerosizer Counter, Coulter Counter, HIAC Counter, or Gelman Automatic Particle Counter) to ascertain the average particle size. Scanning electron microscopy can be utilized to qualitatively assess particle morphology.

Similarly, the powder particles of the invention may be characterized on the basis of density or a range of particle densities. Actual particle density or “absolute density” can be readily ascertained using known quantification techniques such as helium pycnometry and the like. Alternatively, “tap” density measurements can be used to assess the density of a powder according to the invention. Tap density is defined as the mass of a material that upon packing in a specified manner fills a container to a specific volume, divided by the container volume. Suitable devices are available for determining tap density, for example, the GeoPyc™ Model 1360, available from the Micromeritics Instrument Corp. The difference between the absolute density and tap density of a powder composition provides information about the composition's percentage total porosity and specific pore volume.

The average particle size of the powder formulations made in accordance with the present methods is generally about 35 μm to about 300 μm, particularly about 80 μm to about 250 μm, more particularly about 80 μm to about 100 μm. In some embodiments of the invention, the average particle size is at least 80 μm. The average tap density of the powder particles of the invention is typically about 0.01 to about 0.7 g/cm³, particularly about 0.01 to about 0.6 g/cm³, more particularly about 0.02 to about 0.4 g/cm³.

Pharmaceutical compositions comprising a stable powder formulation of an alum-adsorbed vaccine, particularly an alum-adsorbed hepatitis B, botulism (e.g., BoNT/A), anthrax (i.e., B. anthracis), rSEB (i.e., Staphylococcal enterotoxin), or plague (Y. pestis) vaccine are also encompassed by the present invention. In certain embodiments, the pharmaceutical composition comprises a stable powder formulation of a hepatitis B antigen (e.g., HBsAg), a BoNT immunogen (e.g., BoNT/A, particularly BoNT/A HC), a B. anthracis antigen (e.g., B. anthracis PA, particularly B. anthracis rPA), a Staphylococcal enterotoxin antigen (e.g., SEB, particularly rSEB), or a Y. pestis antigen (e.g., F1-V) adsorbed onto an aluminum adjuvant (e.g., aluminum hydroxide). While not intending to limit the invention to specific formulations, in certain embodiments the dried powder comprises (by percent weight) from about 0.0001% to about 10% immunogen, from about 0.2 to about 25% alum adjuvant (based on elemental aluminum content), and from about 70 to about 99% carbohydrate excipient (e.g., mannitol, trehalose, or dextran). Pharmaceutical compositions of the invention further include stable alum-adsorbed vaccine powder formulations that have been reconstituted in a diluent to form a liquid vaccine for administration to a subject. Methods for producing a reconstituted liquid alum-adsorbed vaccine are further encompassed by the present invention. In certain embodiments, the methods comprise reconstituting a dried powder formulation an alum-adsorbed vaccine of the invention in a pharmaceutically acceptable carrier, as defined herein below.

As described above, a “stable” powder formulation of an alum-adsorbed vaccine is one in which the dried powder particles can be reconstituted in a diluent to produce a reconstituted liquid vaccine that exhibits little or no particle agglomeration, shows no significant decrease in immunogen concentration, retains immunogenicity, maintains antigenicity, and/or exhibits protective efficacy, particularly relative to a liquid formulation of the vaccine that has not been SFD or ASFD and subsequently reconstituted prior to administration to a subject. These parameters can be assessed using a variety of techniques known in the art and described in the experimental examples below (e.g., sedimentation assays, AUSYME assays, analysis of serum antibody concentration, percent survival rates following lethal challenge with a disease-causing pathogen or toxin such as BoNT/A or Y. pestis, and an anthrax lethal toxin neutralization assay). Results obtained with the reconstituted liquid vaccine may be compared with those obtained using the original liquid formulation comprising the immunogen adsorbed onto an aluminum adjuvant (i.e., the original liquid formulation prior to atomizing).

In some embodiments, the reconstituted liquid alum-adsorbed vaccine exhibits little or no particle agglomeration, particularly relative to that of the original liquid formulation. Particle agglomeration may be assessed using such methods as microscopy or sedimentation rate analysis. “Little or no particle agglomeration relative to that of the liquid formulation” indicates, for example, that no significant increase in sedimentation rate is observed with the reconstituted liquid vaccine compared to the sedimentation rate of the original liquid formulation. Furthermore, in certain aspects of the invention, the reconstituted liquid vaccine shows no significant decrease in immunogen concentration relative to the immunogen concentration of the liquid formulation prior to atomization. “No significant decrease in immunogen concentration” is intended to mean that the reconstituted liquid vaccine retains at least about 50, 60, or 70% of the original immunogen concentration, more preferably at least about 80, 85, or 90% of the original immunogen concentration, most preferably at least about 91, 92, 93, 94, 95, 96, 97, 98, 99% or more of the immunogen concentration present in the original liquid formulation. Immunogen concentration may be measured, for example, by an ELISA-based method (e.g., AUSZYME).

As used herein and defined in the art, “antigenicity” is the ability of an antibody to recognize and bind to a protein (e.g., an immunogen). “Immunogenicity” refers to the ability of the protein (i.e., immunogen) to raise an immune response in vivo (e.g., in a human or non-human subject). The reconstituted liquid alum-adsorbed vaccines of the invention generally retain a substantial level of antigenicity and immunogenicity as compared with that of the original liquid formulation. A “substantial level of antigenicity” is intended to mean that the immunogen present in the liquid reconstituted vaccine retains at least about 50, 60, 70, 80, 85, 90, 95, 99% or more antigenicity when compared with that of the original liquid vaccine formulation. Antigenicity can be measured by, for example, an ELISA-based assay such as AUSZYME. In certain aspects of the invention, the reconstituted liquid vaccine retains a substantial level of immunogenicity and is therefore able to stimulate an immune response in a subject, particularly an immune response that is substantially the same as that obtained with the original liquid formulation prior to atomization. That is, the immune response achieved by immunization of a subject with the reconstituted liquid vaccine may be greater than, equal to, or at least about 50, 60, 70, 80, 85, 90, 95, 99% or more of the level of immune response obtained with the liquid formulation. Immune response in a subject may be determined by a variety of methods known in the art, including but not limited to measuring serum antibody levels following immunization. Moreover, the “level of protection” or “protective efficacy” obtained with a vaccine of the invention, particularly a vaccine comprising BoNT/A, rPA, rSEB, F1-V any combination thereof, may be assessed by the percentage of immunized subjects surviving following exposure to a lethal dose of an immunogen of interest (e.g., a BoNT such as BoNT/A). The level of protection obtained with an anthrax vaccine of the invention, particularly a B. anthracis rPA vaccine, may be determined by, for example, quantifying the neutralizing antibody titer in serum sample, in accordance with methods known in the art and described in Example 5.

The term “stable” as applied to the powder compositions herein further indicates that the powders may be subjected to high temperatures, long-term storage, or freeze-thaw cycles and still retain the desired properties with respect to agglomeration, immunogen concentration, immunogenicity, antigenicity, and/or protective efficacy described above.

As discussed above, a stable powder formulation of an alum-adsorbed vaccine may be reconstituted in a pharmaceutically acceptable carrier to produce a liquid vaccine formulation suitable for administration to a subject. A “pharmaceutically acceptable carrier” refers to a carrier that is conventionally used in the art to facilitate the storage, administration, or the therapeutic effect of the active ingredient. Pharmaceutically acceptable carriers and methods for formulating pharmaceutical compositions and vaccines are generally known in the art. A thorough discussion of formulation and selection of pharmaceutically acceptable carriers, stabilizers, and isomolytes can be found in Remington's Pharmaceutical Sciences (18^{th} ed.; Mack Publishing Company, Eaton, Pa., 1990), herein incorporated by reference. Exemplary pharmaceutically acceptable carriers for reconstitution of vaccine powder formulations include a variety of diluents such as physiological saline, buffers, and salts. The terms “reconstituted liquid vaccine” or “reconstituted alum-adsorbed liquid vaccine” are used herein interchangeably to refer to pharmaceutical compositions comprising a stable powder formulation of an alum-adsorbed vaccine that has been reconstituted in a liquid carrier to produce a reconstituted liquid vaccine. The methods of the present invention enable the preparation of a dry powder vaccine formulation that is stable and can be readily reconstituted. In particular embodiments, the reconstituted liquid vaccines of the invention exhibit little or no particle agglomeration, display no significant decrease in immunogen concentration, and retain a substantial level of immunogenicity, antigenicity, and/or protective efficacy.

The pharmaceutical compositions of the invention find use in methods of preventing or treating a disease, disorder, condition, or symptoms associated with a particular immunogen. The terms “disease,” “disorder,” and “condition” will be used interchangeably herein. Specifically, the prophylactic and therapeutic methods comprise administration of a therapeutically effective amount of a pharmaceutical composition to a subject. In particular embodiments, methods for preventing or treating hepatitis B are provided. In other aspects of the invention, methods of preventing botulism or the development of the symptoms associated with exposure to a BoNT are further provided. Methods for preventing or treating anthrax are also disclosed. As used herein, “preventing” a disease or disorder is intended administration of a therapeutically effective amount of a pharmaceutical composition of the invention, such as a reconstituted liquid vaccine, to a subject in order to protect the subject from the development of the particular disease or disorder associated with the immunogen, or the symptoms thereof. In some embodiments, a vaccine composition of the invention is administered to a subject such as a human that is at risk for developing the disease or symptoms thereof, particularly hepatitis B, botulism, or anthrax. Methods of preventing the development of symptoms associated with exposure to a BoNT (e.g., blurred vision, dysphagia, respiratory paralysis, musculoskeletal paralysis, cardiac or respiratory arrest, etc.) are also disclosed. Methods of preventing anthrax or the development of symptoms associated with exposure to B. anthracis (e.g., high fever, chest pain, oxygen depletion, secondary shock, increased vascular permeability, systemic hemorrhagic pathology, cardiac or respiratory arrest, etc.) are further encompassed by the present invention. Methods of preventing the development of symptoms associated with exposure, particularly inhalational exposure, to a Staphylococcal enterotoxin and methods for treating a subject exposed to this agent are also disclosed. Methods of preventing plague or the development of symptoms associated with exposure to Y. pestis in a subject as well as methods of treating a subject with the plague or exposed to Y. pestis are further envisioned in the present invention. Vaccines of the invention directed to potential bioterrorist agents, including but not limited to a BoNT, B. anthracis, Y. pestis, and a Staphylococcal enterotoxin, may be prepared, for example, as polyvalent vaccines or administered prophylactically to first responders and military personnel or even to the general population in response to a bioterrorist event or threatened bioterrorist event.

By “treating a disease or disorder” is intended administration of a therapeutically effective amount of a pharmaceutical composition of the invention to a subject that is afflicted with the disease or that has been exposed to a pathogen that causes the disease, where the purpose is to cure, heal, alleviate, relieve, alter, remedy, ameliorate, improve, or affect the condition or the symptoms of the disease.

A “therapeutically effective amount” refers to an amount that provides a therapeutic effect for a given condition and administration regimen. In particular aspects of the invention, a “therapeutically effective amount” refers to an amount of a pharmaceutical composition of the invention that when administered to a subject brings about a positive therapeutic response with respect to the prevention or treatment of a subject for a disease. A positive therapeutic response with respect to preventing a disease includes, for example, eliciting an immune response (e.g., the production of antibodies by the subject in a quantity sufficient to protect against development or progression of the disease). Similarly, a positive therapeutic response in regard to treating a disease includes curing or ameliorating the symptoms of the disease.

A therapeutically effective amount can be determined by the ordinary skilled medical worker based on patient characteristics (age, weight, sex, condition, complications, other diseases, etc.). Moreover, as further routine studies are conducted, more specific information will emerge regarding appropriate dosage levels for treatment of various conditions in various patients, and the ordinary skilled worker, considering the therapeutic context, age and general health of the recipient, will be able to ascertain proper dosing. The therapeutically effective amount will be further influenced by the route of administration of the pharmaceutical composition. Generally, for intravenous injection or infusion, and particularly for intradermal administration, the therapeutically effective amount may be lower than that required for intraperitoneal, intramuscular, intranasal, or other route of administration. The dosing schedule may vary, depending on the circulation half-life, and the formulation used. Precise amounts of the pharmaceutical composition required to be administered will depend on the judgment of the practitioner and are peculiar to each individual.

The vaccines of the invention are administered in a manner compatible with the dosage formulation and in such amount as are therapeutically effective and immunogenic (i.e., an antibody-inducing or protective amount, as is desired). The quantity to be administered depends on the subject to be treated, capacity of the subject's immune system to synthesize antibodies, and degree of protection desired. Precise amounts of immunogen required to be administered depend on the judgment of the practitioner and are peculiar to each individual. In a protein vaccine, the amount of protein in each vaccine dose is selected as an amount which induces an immunoprotective response without significant, adverse side effects in typical vaccines. Such amount will vary depending upon which specific immunogen is employed and how it is presented. Optimal amounts of components for a particular vaccine can be ascertained by standard studies involving observation of appropriate immune responses in subjects. Following an initial vaccination, subjects may receive one or several booster immunizations adequately spaced.

The pharmaceutical compositions of the invention can be administered to a subject by a variety of methods known in the art. Any method for administering a composition to a subject may be used in the practice of the invention. Examples of possible routes of administration include pulmonary inhalation, parenteral administration (e.g., intravenous (IV), intramuscular (IM), intradermal (ID), intraperitoneal (IP), subcutaneous (SC) injection or infusion), oral, intranasal, transdermal (topical), transmucosal, and rectal administration. As described above, a pharmaceutical composition comprising an alum-adsorbed vaccine may be administered as a stable powder (e.g., by pulmonary inhalation, intranasal delivery, or transdermal injection) or as a reconstituted powder vaccine formulation (e.g. by intradermal, intramuscular, or intravenous injection). In particular, the pharmaceutical compositions comprising a stable powder formulation of an alum-adsorbed vaccine may be suitable for administration to a subject in powder form by, for example, intranasal delivery, pulmonary inhalation, or transdermal injection. Alternatively, reconstituted liquid vaccines may be administered, for example, intradermally, intravenously, intramuscularly, subcutaneously, intraperitoneally, or intranasally. In certain embodiments of the invention, the vaccines are administered via a minimally invasive method, such as, for example, by intradermal injection through a microneedle or by intranasal inhalation. As used herein, “microneedle” typically includes needles that are 30-gauge or smaller, particularly a 34-gauge needle. Such minimally invasive methods of vaccine administration may permit widespread vaccine administration to the general population by non-medical personnel, which would be particularly advantageous in the event or threat of a bioterrorist attack with a biological weapon such as a BoNT, B. anthracis, Y. pestis, or a Staphylococcal enterotoxin (e.g., rSEB).

The prophylactic and therapeutic methods of the present invention are not intended to be limited to particular subjects. A variety of subjects, particularly mammals, are contemplated. Subjects of interest include but are not limited to humans, dogs, cats, horses, pigs, cows, and rodents. In particular embodiments, the subject is a human, more particularly a human patient at risk for developing the disease associated with the specific antigen.

In accordance with the present invention there may be employed conventional molecular biology, microbiology, and recombinant DNA techniques within the skill of the art. Such techniques are explained fully in the literature. See, e.g., Sambrook et al., Molecular Cloning: A Laboratory Manual (1989); Current Protocols in Molecular Biology, Volumes I-III (Ausubel, R. M., ed. (1994)); Cell Biology: A Laboratory Handbook, Volumes I-III (J. E. Celis, ed. (1994)); Current Protocols in Immunology, Volumes I-III (Coligan, J. E., ed. (1994)); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The following examples are offered by way of illustration and not by way of limitation.

### EXPERIMENTAL

### Example 1

### Preparation of Powder Formulations of an Alum-Adsorbed Hepatitis B Vaccine

### Spray-Freeze-Drying (SFD)

Powder formulations of the Shanvac-B hepatitis B vaccine (Shanvac-B, Shantha Biotechnics, Hyderabad, India) were produced by SFD essentially as described for the recombinant Protective Antigen of Bacillus anthracis (rPA) anthrax vaccine in Jiang et al. (2006) J. Pharm. Sci. 95:80-96. The Shanvac-B vaccine comprises recombinant HBsAg adsorbed onto aluminum hydroxide adjuvant. Briefly, a liquid formulation comprising Shanvac-B vaccine and dextran/trehalose or mannitol/trehalose was prepared and sprayed using a BD Accuspray® nozzle affixed to a 5 ml syringe. 5 ml aliquots were sprayed into a metal pan containing liquid nitrogen, and the pan was transferred to a shelf lyophilizer pre-cooled to −40 C. The liquid nitrogen was allowed to completely evaporate, and the powder was then dried as described in Table 1 below.

TABLE 1
Drying Conditions for SFD Method

| Time | Temperature | Pressure |
|---|---|---|
| 6 hours | −40° C. | 50 mT |
| 12 hours | −20° C. | 50 mT |
| 9 hours | 0° C. | 50 mT |
| 12 hours | 10° C. | 50 mT |
| 3 hours | 20° C. | 50 mT |

The powder vaccine samples were removed from the lyophilizer, placed in a dry glove box (5% relative humidity (“RH”)), and then transferred into glass containers and sealed.

### Atmospheric Spray-Freeze-Drying (ASFD)

Powder formulations of the Shanvac-B hepatitis B vaccine (Shanvac-B, Shantha Biotechnics, Hyderabad, India) were produced by ASFD essentially as described in U.S. Patent Application Publication No. 2003/0180755. Specifically, a liquid formulation comprising the Shanvac-B hepatitis B vaccine and mannitol/trehalose was prepared. A syringe was charged with this formulation and sprayed using an ultrasonic nozzle (Sono-Tek Model 8700-25; operating a 4.5 watts and a liquid flow rate of 6 ml/min) into an ASFD chamber filled with liquid nitrogen. When the spraying was completed, a dry nitrogen gas flow was initiated into the bottom of the chamber at a rate of 15 L/min. The dry nitrogen gas flow continued until all of the liquid nitrogen evaporated. The inlet gas flow was then increased to 100 L/min for 2.5 hours to anneal the powder at −30° C. prior to drying. The gas flow was then reduced to 38 L/min to reach the desired drying temperature of −14° C. When the measured percent RH dropped to less than 1%, the chiller was turned off, keeping the gas flow at 38 L/min. The chamber was allowed to warm gradually to 20° C., at which point the gas flow was shut off and the powder harvested. The powder yield (determined gravimetrically) was 87.7%.

### Example 2

### Analysis of SFD Hepatitis B Vaccine

### Stability Analysis

The SFD Shanvac-B hepatitis B vaccine prepared as described above in Example 1 and the original liquid formulation of the liquid Shanvac-B vaccine were analyzed for particle agglomeration under various conditions. Specifically, each vaccine formulation (liquid or powder) was stored at 4° C. (as recommended by the manufacturer of the liquid Shanvac-B vaccine), at 55° C., or subjected to a freeze-thaw at −20° C. followed by storage at 55° C. The SFD powder formulation of the hepatitis B vaccine was then reconstituted in water and subjected to further analysis. In particular, the stability of the vaccine formulations following the various storage conditions was assessed using sedimentation and AUSYME assays, as described below.

### Sedimentation Assay

Approximately 250 μl of sample was drawn up into a glass capillary tube, sealed with CRITOSEAL, and left in an upright position at room temperature (24° C.). The height of the turbid precipitate fraction and the total height of liquid were measured and recorded at the following time points post capillary tube erection: 0 min, 10 min, 20 min, 30 min, 45 min, 1 hr, 2 hr, 3 hr, 4 hr, and 5 hr. A total of 3 replicates per sample condition were performed to ensure the accuracy and reproducibility of the experiment. The sedimentation rate was calculated as follows:

$$\% sedimentation = \frac{( total liquid height - recorded precipitate height )}{total liquid height} \times 100$$

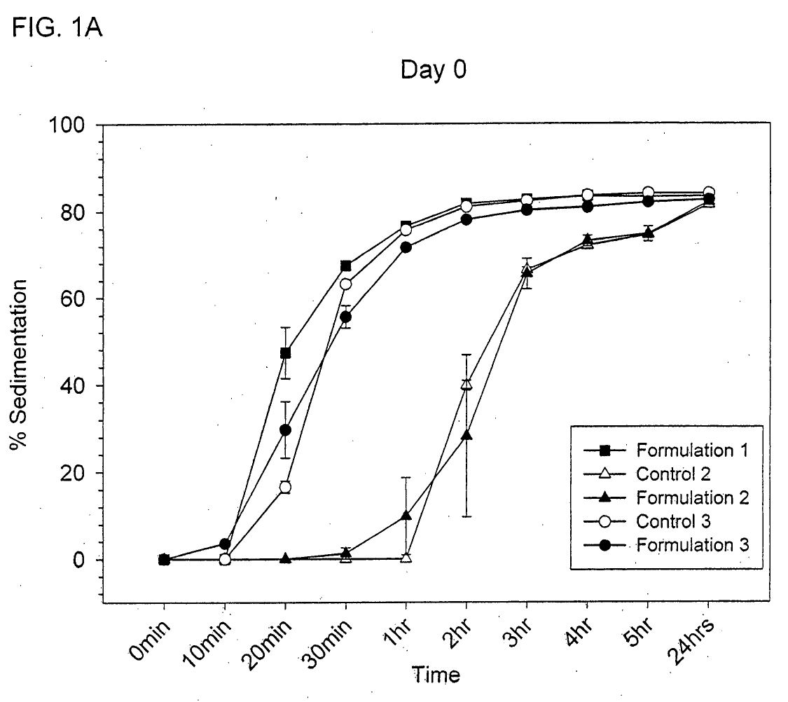

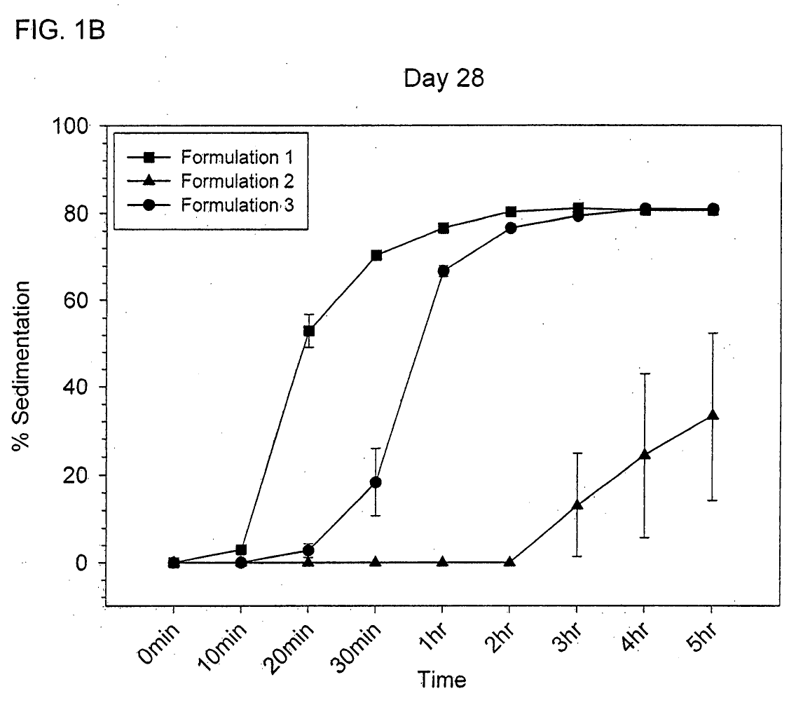

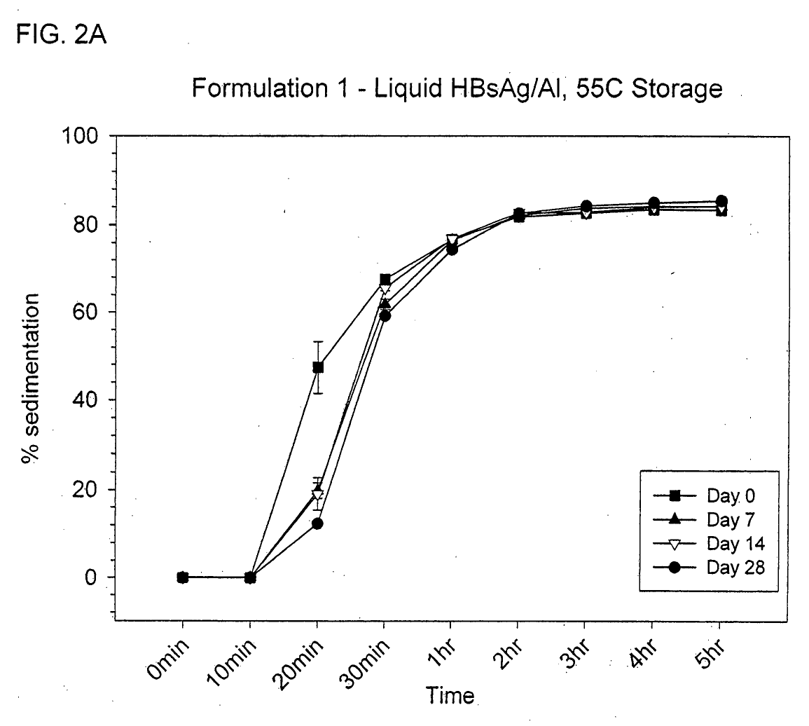

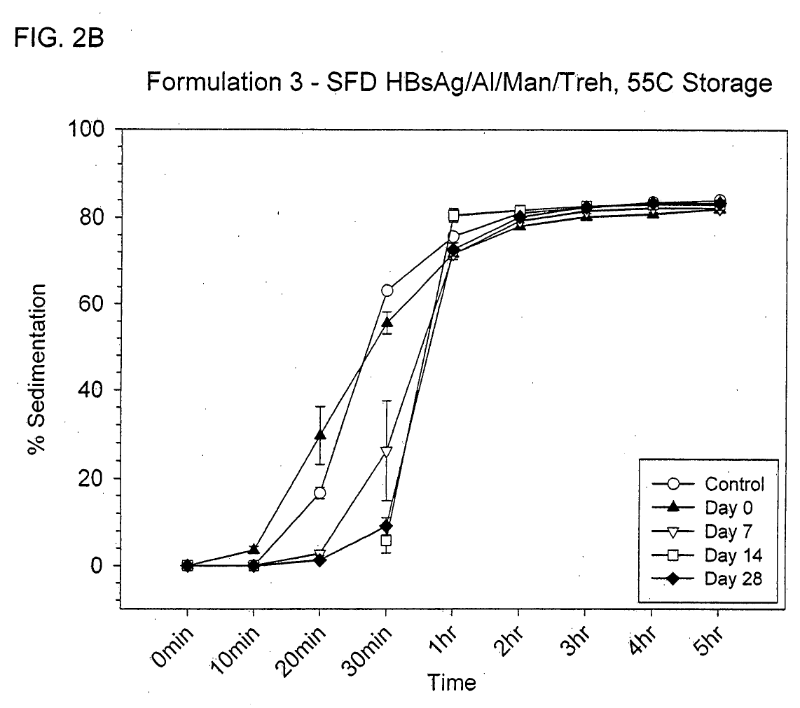

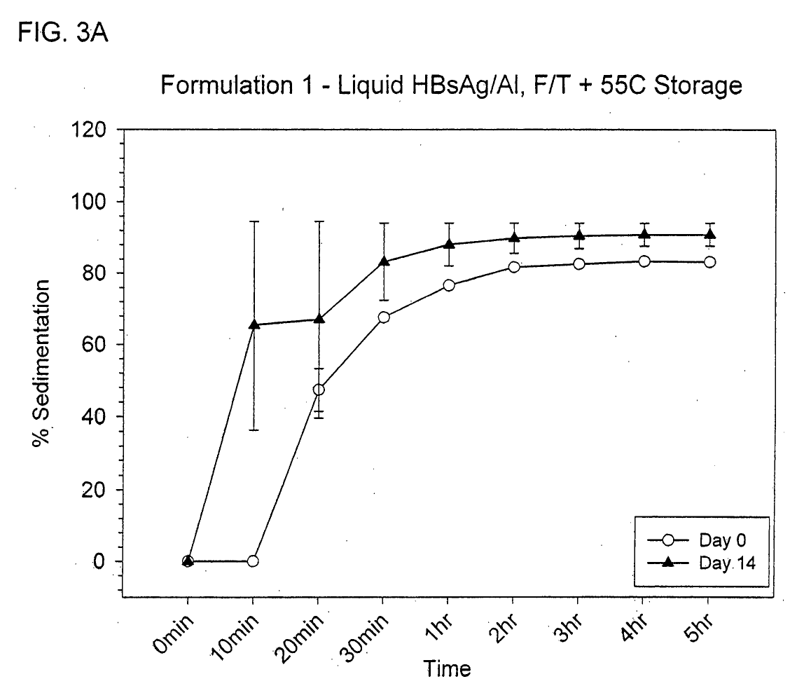

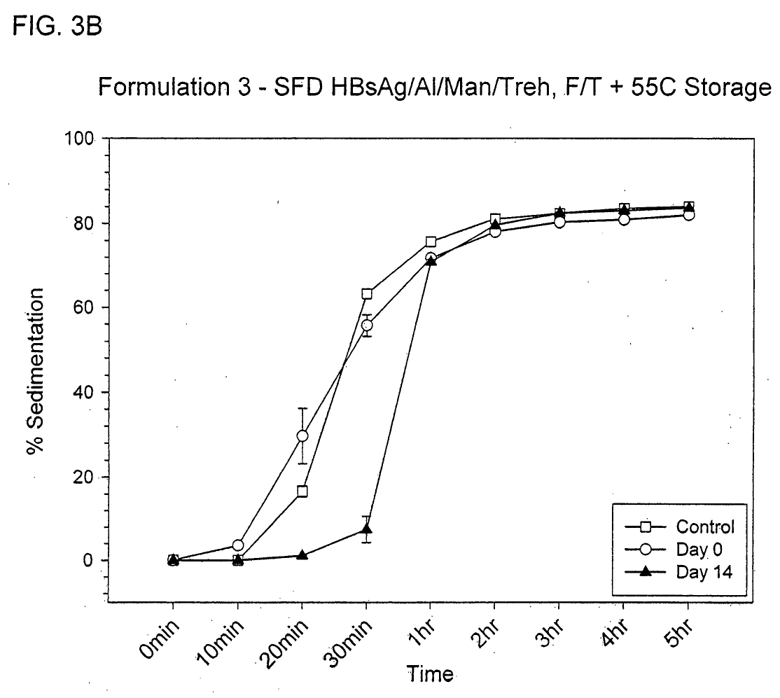

The results obtained in the sedimentation assays are provided in FIGS. 1-3. No significant increase in sedimentation rate (i.e., leftward shift in the sedimentation curve) was observed in samples after SFD processing with either mannitol/trehalose or dextran/trehalose and storage at 4° C. and 55° C. for one month (FIGS. 1 and 2). The sedimentation rate was slowed by addition of dextran, presumably due to an increase in viscosity (FIG. 1). Furthermore, the SFD formulation with mannitol/trehalose showed no evidence of agglomeration after freeze thaw and storage at 55° C. for 14 days (FIG. 3).

In contrast to the SFD vaccine formulation, the original liquid formulation of the vaccine showed a faster sedimentation rate after freeze-thaw and storage at 55° C. for 14 days, which is consistent with particle agglomeration (FIG. 3). Accordingly, SFD processing appears to protect the HBsAg vaccine containing aluminum adjuvant from particle agglomeration.

A summary of the sedimentation data presented in FIGS. 1-3 is provided below in Tables 2-7.

TABLE 2
Summary of Sedimentation Data for Various Shanvac-B Vaccine Formulations
(Day 0)

| | % Sedimentation | | | | |
|---|---|---|---|---|---|
| Time | Formulation 1 | Control 2 | Formulation 2 | Control 3 | Formulation 3 |
| 0 min | 0 | 0 | 0 | 0 | 0 |
| 10 min | 0.0 | 0.0 | 0.0 | 0.0 | 3.6 |
| 20 min | 47.4 | 0.0 | 0.0 | 16.6 | 29.6 |
| 30 min | 67.6 | 0.0 | 1.3 | 63.2 | 55.7 |
| 1 hr | 76.6 | 0.0 | 9.8 | 75.7 | 71.8 |
| 2 hr | 81.7 | 39.8 | 28.2 | 81.0 | 78.1 |
| 3 hr | 82.7 | 66.6 | 65.6 | 82.3 | 80.2 |
| 4 hr | 83.5 | 72.1 | 73.2 | 83.5 | 80.8 |
| 5 hr | 83.3 | 74.6 | 74.8 | 84.0 | 82.0 |

*Note: Formulation 1 (liquid Shanvac-B vaccine); Control 2 (liquid Shanvac-B vaccine + dextran/trehalose); Formulation 2 (SFD Shanvac-B vaccine + dextran/trehalose); Control 3 (liquid Shanvac-B vaccine + mannitol/trehalose); and Formulation 3 (SFD Shanvac-B vaccine + mannitol/trehalose).

TABLE 3
Summary of Sedimentation Data for Various Shanvac-B Vaccine
Formulations (Day 28)

| | | % Sedimentation | | |
|---|---|---|---|---|
| | | Formulation | Formulation | Formulation |
| Time | | 1 | 2 | 3 |
| 0 | min | 0 | 0 | 0 |
| 10 | min | 3.0 | 0.0 | 0.0 |
| 20 | min | 53.0 | 0.0 | 2.8 |
| 30 | min | 70.5 | 0.0 | 18.3 |
| 1 | hr | 76.7 | 0.0 | 66.9 |
| 2 | hr | 80.4 | 0.0 | 76.7 |
| 3 | hr | 81.3 | 13.1 | 79.5 |
| 4 | hr | 80.8 | 24.3 | 81.1 |
| 5 | hr | 80.8 | 33.4 | 81.1 |

*Note:
Formulation 1 (liquid Shanvac-B vaccine);
Control 2 (liquid Shanvac-B vaccine + dextran/trehalose);
Formulation 2 (SFD Shanvac-B vaccine + dextran/trehalose);
Control 3 (liquid Shanvac-B vaccine + mannitol/trehalose); and
Formulation 3 (SFD Shanvac-B vaccine + mannitol/trehalose).

TABLE 4
Summary of Sedimentation Data for Liquid Shanvac-B Vaccine Stored
at 55° C. for 0, 7, 14, and 28 days

% Sedimentation

| Time | | Day 0 | Day 7 | Day 14 | Day 28 |
|---|---|---|---|---|---|
| 0 | min | 0 | 0 | 0 | 0 |
| 10 | min | 0.0 | 0.0 | 0.0 | 0.0 |
| 20 | min | 47.4 | 19.8 | 19.1 | 12.3 |
| 30 | min | 67.6 | 61.8 | 65.5 | 59.1 |
| 1 | hr | 76.6 | 76.4 | 76.8 | 74.4 |
| 2 | hr | 81.7 | 82.1 | 82.7 | 82.5 |
| 3 | hr | 82.7 | 83.7 | 82.8 | 84.2 |
| 4 | hr | 83.5 | 84.0 | 84.0 | 85.0 |
| 5 | hr | 83.3 | 84.0 | 84.2 | 85.4 |

TABLE 5
Summary of Sedimentation Data for SFD
Shanvac-B Vaccine Formulations
Stored at 55° C. for 0, 7, 14, and 28 days

| | % Sedimentation | | | | |
|---|---|---|---|---|---|
| | | | | | Day |
| Time | Control | Day 0 | Day 7 | Day 14 | 28 |
| 0 min | 0 | 0 | 0 | 0 | 0 |
| 10 min | 0.0 | 3.6 | 0.0 | 0.0 | 0.0 |
| 20 min | 16.6 | 29.6 | 2.8 | 0.0 | 0.0 |
| 30 min | 63.2 | 55.7 | 26.2 | 5.8 | 9.1 |
| 1 hr | 75.7 | 71.8 | 71.6 | 80.5 | 72.7 |
| 2 hr | 81.0 | 78.1 | 79.3 | 81.6 | 80.2 |
| 3 hr | 82.3 | 80.2 | 81.5 | 82.6 | 82.4 |
| 4 hr | 83.5 | 80.8 | 82.2 | 83.1 | 83.4 |
| 5 hr | 84.0 | 82.0 | 82.2 | 83.1 | 83.4 |

TABLE 6
Summary of Sedimentation Data for Liquid Shanvac-B Vaccine Following
Freeze-Thaw and Storage at 55° C. for 0 and 14 Days

% Sedimentation

| Time | | Day 0 | Day 14 |
|---|---|---|---|
| 0 | min | 0 | 0 |
| 10 | min | 0.0 | 65.5 |
| 20 | min | 47.4 | 67.0 |
| 30 | min | 67.6 | 83.2 |
| 1 | hr | 76.6 | 88.1 |
| 2 | hr | 81.7 | 89.9 |
| 3 | hr | 82.7 | 90.5 |
| 4 | hr | 83.5 | 90.9 |
| 5 | hr | 83.3 | 90.9 |

TABLE 7
Summary of Sedimentation Data for SFD Shanvac-B Vaccine
Formulations Following Freeze-Thaw and Storage at 55° C. for
0 and 14 Days

| | % Sedimentation | | |
|---|---|---|---|
| Time | Control | Day 0 | Day 14 |

| | | | | |
|---|---|---|---|---|
| 0 | min | 0 | 0 | 0 |
| 10 | min | 0 | 0.0 | 65.5 |
| 20 | min | 16.6 | 47.4 | 67.0 |
| 30 | min | 63.2 | 67.6 | 83.2 |
| 1 | hr | 75.7 | 76.6 | 88.1 |
| 2 | hr | 81.0 | 81.7 | 89.9 |
| 3 | hr | 82.3 | 82.7 | 90.5 |
| 4 | hr | 83.5 | 83.5 | 90.9 |
| 5 | hr | 84.0 | 83.3 | 90.9 |

AUSZYME Assay

The concentration of HBsAg ([HBsAg]) in each sample was quantified by AUSZYME assay (Abbott Laboratories, Abbott Park, Ill.) in accordance with the manufacturer's instructions. Briefly, each sample was diluted 1/2000 in water to a [HBsAg] of 10 ng/ml, before being added in duplicate to wells of the supplied multi-well plate. A standard curve was constructed by diluting Shanvac-B vaccine in two-fold dilutions from 20 ng/ml to 0.625 ng/ml. Monoclonal conjugate was added to each well followed by an anti-HBsAg monoclonal coated bead. After incubation at 37° C. for 75 min, each well containing a bead was washed 3 times with 5 ml of water. OPD substrate (o-Phenylenediamine.2HCl), was then added to each bead and incubated at room temperature for 30 minutes. The reaction was stopped with 1N H₂SO₄, and the absorbance of the supernatant was read by plate reader at OD₄₉₂. [HBsAg] in the SFD processed vaccine and original liquid vaccine samples were determined by comparison to the standard curve.

The results of the AUSYME assays are summarized in Table 8 below.

TABLE 8
Concentration of HBsAg in Liquid and SFD
Hepatitis B Vaccine Formulations
Under Various Storage Conditions

| | | | | Freeze-thaw + | | |
|---|---|---|---|---|---|---|
| | Day 0 | Day 7, 55° C. | Day 14, 55° C. | Day 14, 55° C. | Day 28, 55° C. | Day 28, 4° C. |
| Liquid | 15.21 | 7.31 | 3.17 | 9.96 | 1.5 | 13.94 |
| SFD D/T | 12.2 | 8.94 | 8.79 | 8.85 | 6.93 | 9.84 |
| SFD M/T | 12.66 | 13.08 | 11.71 | 10.03 | 10.29 | 15.84 |

*D/T = dextran/trehalose; M/T = mannitol/trehalose

AUSZYME assay results demonstrated that the liquid Shanvac-B vaccine, while stable at 4° C., showed a decrease in [HBsAg] after 14 days at 55° C., with a further drop in antigen concentration at 28 days. In contrast, SFD processed Shanvac-B vaccine containing mannitol/trehalose excipients retained approximately the same [HBsAg] when stored at both 4° C. and 55° C. for 28 days. The SFD dextran/trehalose formulation did show a decrease in [HBsAg] following storage at 55° C. for 28 days. This decrease, however, was smaller than that for the liquid vaccine under the same storage conditions. Both the liquid and powder formulations retained [HBsAg] when subjected to a freeze-thaw cycle followed by storage at 55° C. for 28 days. This result is surprising considering that the liquid formulation experienced agglomeration at this storage condition as measured by sedimentation assays and considering that the [HBsAg] of the liquid vaccine dropped markedly following storage at 55° C. over 28 days.

### Immunogenicity Studies

To evaluate whether the SFD hepatitis B vaccine formulations retained their immunogenicity, mice were immunized with various formulations of the liquid or reconstituted SFD Shanvac-B vaccine. The immune response was measured by quantifying the serum HBsAg-specific antibody response, as detailed below. Specifically, female Balb/c mice (10 per group), were immunized at day 0 and at day 28 by intramuscular injection with either a 0.4 or 2 microgram dose of one of the following vaccine formulations:

1. Shanvac-B vaccine (liquid)
2. Shanvac-B vaccine+dextran/trehalose (liquid)
3. Shanvac-B vaccine+mannitol/trehalose (liquid)
4. SFD Shanvac-B+dextran/trehalose (reconstituted)
5. SFD Shanvac-B+mannitol/trehalose (reconstituted)
6. Engerix-B vaccine (liquid)

The mice were bled at days 0, 28 and 42. The serum was quantified for antibody to HBsAg by ELISA. Serum samples, diluted in PBS/0.05% Tween-20/1% Nonfat Dry Milk (pH 7.2), were added to the wells of a 96 well Maxisorp plate (Nalgene NUNC, Rochester, N.Y.), previously coated with 0.5 μg/ml HBsAg in phosphate coating buffer. A standard curve was generated using known concentrations of monoclonal anti-HBsAg antibody. Following incubation at room temperature for 1 hour, the plates were washed 3 times with PBS+0.05% Tween-20 (PBST). An anti-mouse IgG conjugate (Southern Biotechnology Associates Inc., Birmingham, Ala.) diluted to 1:8000 in PBST was added, and the plates were incubated at room temperature for 30 minutes. The plates were again washed 3 times with PBST. TMB substrate (3,3′,5,5′-Tetramethylbenzidine substrate) was added, and the plates were incubated at room temperature for 10 minutes. The reaction was stopped by the addition of 1N H₂SO₄, and the plates were read at OD₄₉₂.

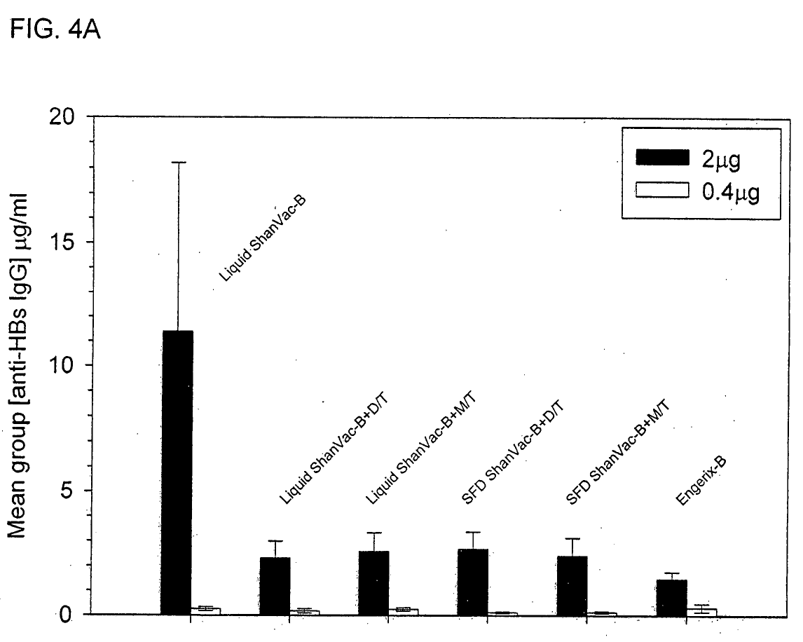

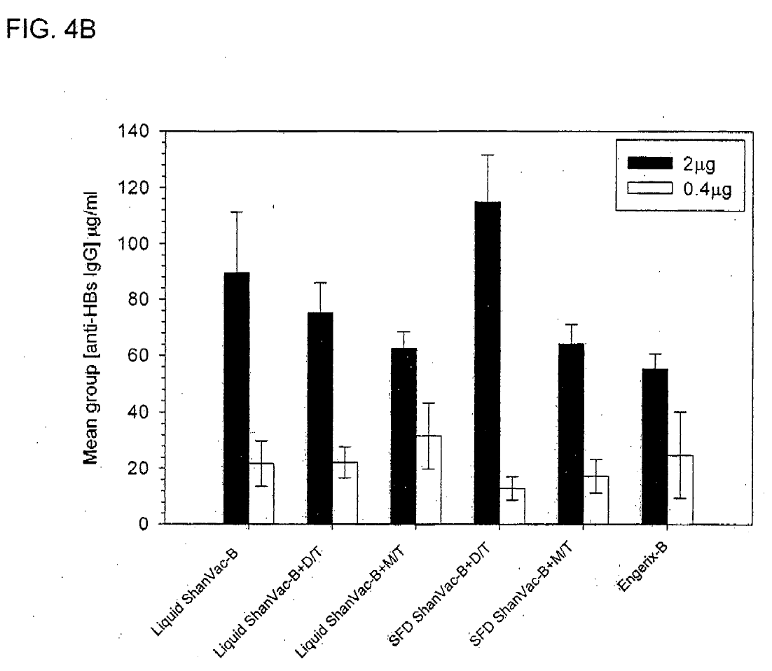

The results of the immunogenicity studies are summarized in FIG. 4 and in Tables 9 and 10 below. For all groups tested, IgG response was dose dependent. The addition of excipient to the liquid vaccine formulation did not significantly affect IgG response. Immunization of mice with reconstituted vaccine powder formulations resulted in antibodies at levels similar to those obtained following immunization with conventional liquid vaccine (FIG. 4). After the second immunization, the antibody levels generated by the SFD formulation were not significantly different than those obtained following immunization with the liquid formulations (p>0.05). These results demonstrate that the SFD process does not affect the immunogenicity of the Shanvac-B hepatitis B vaccine. Therefore, SFD can be used to produce a stable alum-adsorbed hepatitis B vaccine powder formulation that retains immunogenicity upon reconstitution in a diluent.

TABLE 9
Summary of Mean Anti-HBsAg Antibody Concentrations (μg/ml) Serum From Mice Immunized
with Liquid or SFD Hepatitis B Vaccines (28 Days Post-Immunization)

| | | Dose of HBsAg | | | | | | | | | | | | |
|---|---|---|---|---|---|---|---|---|---|---|---|---|---|---|
| | | 2 ug | | | | | | 0.4 ug | | | | | | 0 ug |
| | | Formulation | | | | | | | | | | | | |
| | | Liquid | | | SFD | | Engerix-B | Liquid | | | SFD | | Engerix-B | N/A |
| | | Excipient | | | | | | | | | | | | |
| | | N/A | T/D | T/M | T/D | T/M | N/A | N/A | T/D | T/M | T/D | T/M | N/A | N/A |
| | | Group # | | | | | | | | | | | | |
| | | 1 | 2 | 3 | 4 | 5 | 6 | 7 | 8 | 9 | 10 | 11 | 12 | 13 |
| Individual # | 1 | 2.11 | 1.36 | 8.80 | 5.32 | 0.73 | 0.52 | 0.42 | 0.00 | 0.00 | 0.27 | 0.17 | 0.00 | 0.00 |
| | 2 | 0.77 | 1.29 | 2.48 | 1.63 | 5.01 | 3.12 | 0.00 | 0.00 | 0.13 | 0.00 | 0.23 | 0.37 | 0.00 |
| | 3 | 1.10 | 2.16 | 1.53 | 1.18 | 2.20 | 2.23 | 0.21 | 0.41 | 0.48 | 0.25 | 0.00 | 0.00 | 0.00 |
| | 4 | 2.53 | 1.51 | 0.54 | 1.37 | 1.33 | 2.39 | 0.19 | 0.26 | 0.16 | 0.00 | 0.00 | 0.65 | 0.00 |
| | 5 | 1.20 | 1.79 | 1.47 | 0.56 | 0.33 | 0.75 | 0.00 | 0.00 | 0.42 | 0.18 | 0.00 | 1.73 | 0.00 |
| | 6 | 50.13 | 4.65 | 1.21 | 7.01 | 2.47 | 1.17 | 0.00 | 0.27 | 0.54 | 0.00 | 0.00 | 0.00 | 0.00 |
| | 7 | 54.30 | 0.32 | 3.13 | 1.55 | 0.08 | 0.84 | 0.42 | 0.00 | 0.26 | 0.00 | 0.17 | 0.00 | 0.00 |
| | 8 | 0.00 | 1.01 | 1.41 | 0.84 | 0.84 | 0.47 | 0.79 | 0.00 | 0.00 | 0.29 | 0.00 | 0.00 | 0.00 |
| | 9 | 0.84 | 1.62 | 3.39 | 3.06 | 6.17 | 1.70 | 0.41 | 0.00 | 0.00 | 0.00 | 0.39 | 0.18 | 0.00 |
| | 10 | 0.94 | 7.40 | 1.81 | 4.16 | 5.00 | 1.50 | 0.18 | 0.82 | 0.52 | 0.18 | 0.31 | 0.15 | 0.00 |
| | Group Mean | 11.39 | 2.31 | 2.58 | 2.67 | 2.41 | 1.47 | 0.26 | 0.18 | 0.25 | 0.12 | 0.13 | 0.31 | 0.00 |

TABLE 10
Summary of Mean Anti-HBsAg Antibody Concentrations in Serum from Mice Immunized
with Liquid or SFD Hepatitis B Vaccines (42 Days Post-Immunization)

| | | Dose of HBsAg | | | | | | | | | | | | |
|---|---|---|---|---|---|---|---|---|---|---|---|---|---|---|
| | | 2 ug | | | | | | 0.4 ug | | | | | | 0 ug |
| | | Formulation | | | | | | | | | | | | |
| | | Liquid | | | SFD | | Engerix-B | Liquid | | | SFD | | Engerix-B | N/A |
| | | Excipient | | | | | | | | | | | | |
| | | N/A | T/D | T/M | T/D | T/M | N/A | N/A | T/D | T/M | T/D | T/M | N/A | N/A |
| | | Group # | | | | | | | | | | | | |
| | | 1 | 2 | 3 | 4 | 5 | 6 | 7 | 8 | 9 | 10 | 11 | 12 | 13 |
| Individual # | 1 | 64.25 | 112.79 | 59.65 | 179.73 | 32.33 | 56.34 | 87.56 | 16.79 | 12.64 | 13.77 | 9.55 | 9.4022909 | 0.04 |
| | 2 | 28.05 | 88.27 | 34.52 | 104.26 | 41.10 | 59.75 | 15.03 | 19.19 | 27.05 | 4.41 | 13.21 | 10.373883 | 0.04 |
| | 3 | 42.14 | 139.02 | 64.27 | 88.94 | 66.95 | 70.60 | 41.99 | 25.62 | 16.62 | 21.90 | 3.78 | 15.266531 | 0.04 |
| | 4 | 77.68 | 80.32 | 56.34 | 198.43 | 58.51 | 85.10 | 15.72 | 26.41 | 19.93 | 2.61 | 12.77 | 11.991956 | 0.05 |
| | 5 | 93.28 | 37.39 | 66.97 | 76.20 | 78.41 | 48.22 | 5.78 | 0.46 | 11.20 | 12.86 | 5.79 | 162.395 | 0.03 |
| | 6 | 192.47 | 52.22 | 90.66 | 140.12 | 73.26 | 51.58 | 4.43 | 21.61 | 35.41 | 2.53 | 7.93 | 6.178284 | 0.04 |
| | 7 | 129.48 | 34.03 | 44.49 | 38.38 | 40.57 | 57.19 | 7.86 | 9.65 | 23.78 | 4.07 | 9.29 | 1.1083183 | 0.04 |
| | 8 | 11.28 | 56.36 | 42.62 | 52.70 | 86.82 | 30.84 | 20.55 | 20.41 | 16.79 | 45.70 | 7.59 | 4.452978 | 0.03 |
| | 9 | 212.36 | 58.15 | 83.89 | 135.66 | 98.64 | 31.31 | 8.79 | 14.58 | 17.70 | 13.36 | 63.21 | 19.616255 | 0.04 |
| | 10 | 45.37 | 93.60 | 83.44 | 135.26 | 67.49 | 63.58 | 9.30 | 66.68 | 134.70 | 8.25 | 39.64 | 7.9996836 | 0.01 |
| | Group | 89.64 | 75.22 | 62.68 | 114.97 | 64.41 | 55.45 | 21.70 | 22.14 | 31.58 | 12.94 | 17.27 | 24.88 | 0.03 |
| | Mean | | | | | | | | | | | | | |

### Example 3

### Analysis of ASFD Hepatitis B Vaccine

### Stability Analysis

The ASFD Shanvac-B hepatitis B vaccine prepared as described above in Example 1 and the original liquid formulation of the liquid Shanvac-B vaccine were analyzed for particle agglomeration under various conditions. Specifically, each vaccine formulation (liquid or powder) was stored at 4° C. (as recommended by the manufacturer of the liquid Shanvac-B vaccine), at 55° C., or subjected to a freeze-thaw at −20° C. followed by storage at 55° C. The ASFD powder formulation of the hepatitis B vaccine was then reconstituted in water and subjected to further analysis. In particular, the stability of the vaccine formulations following the various storage conditions was assessed using sedimentation and AUSYME assays, as described above in Example 2.

### Sedimentation Assay

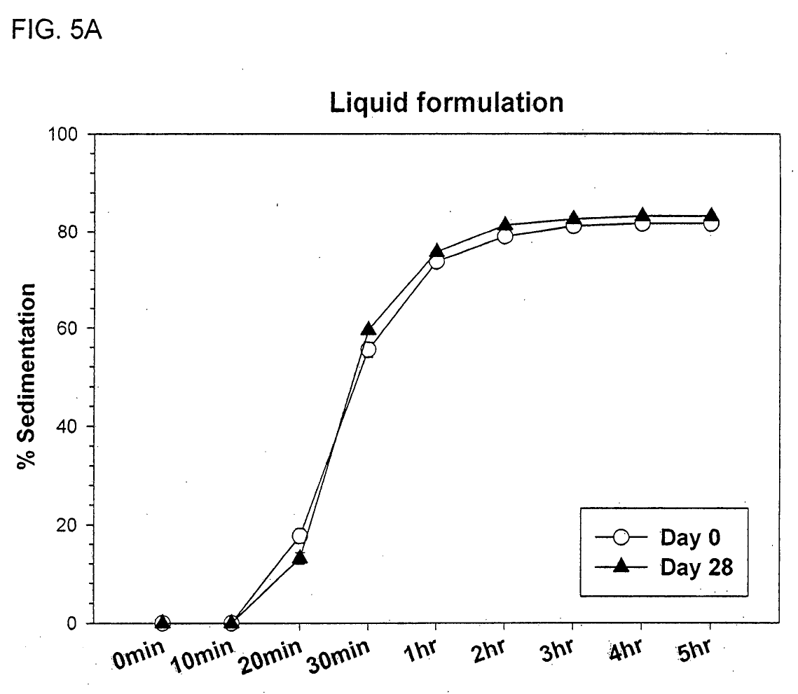

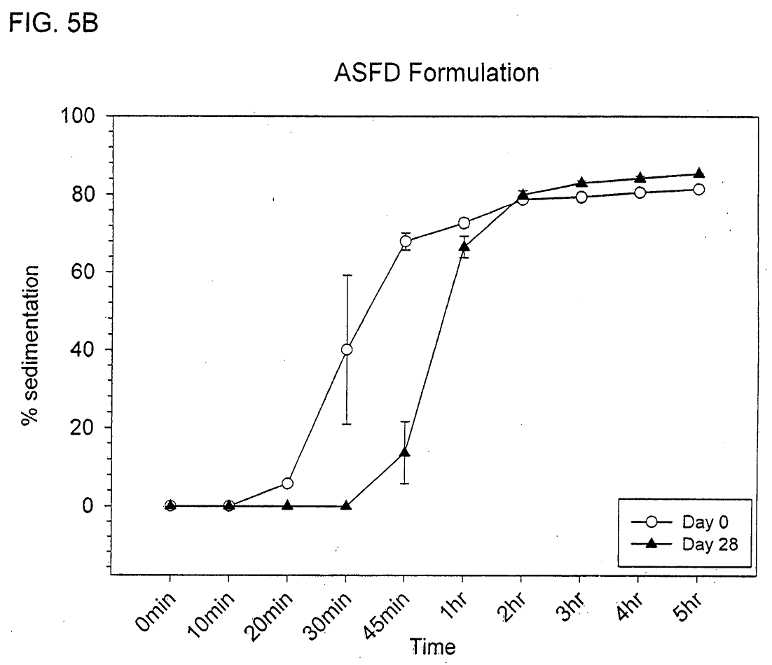

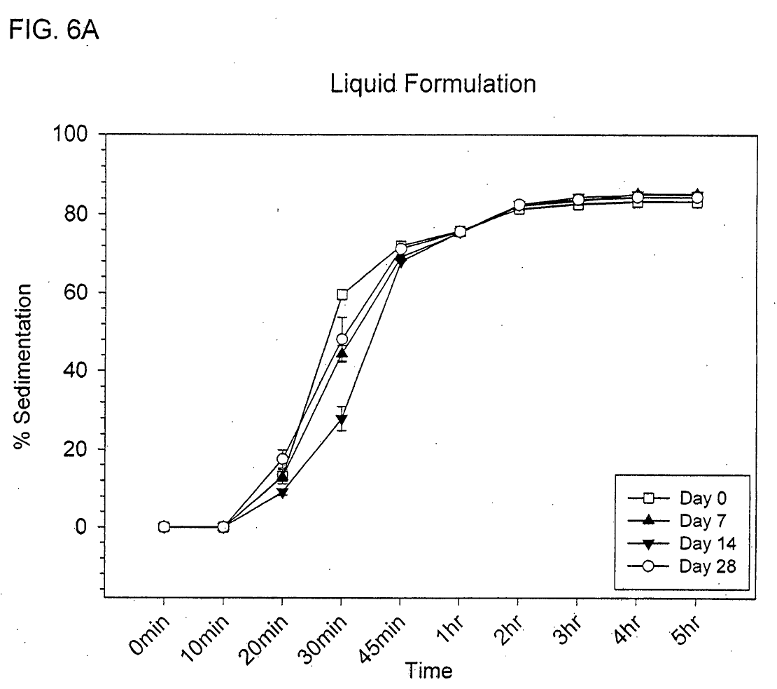

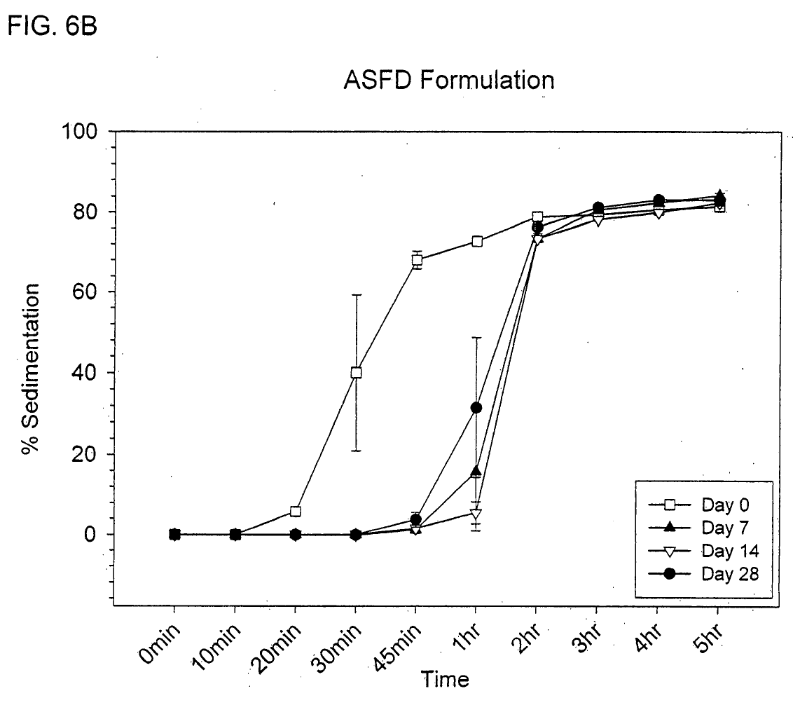

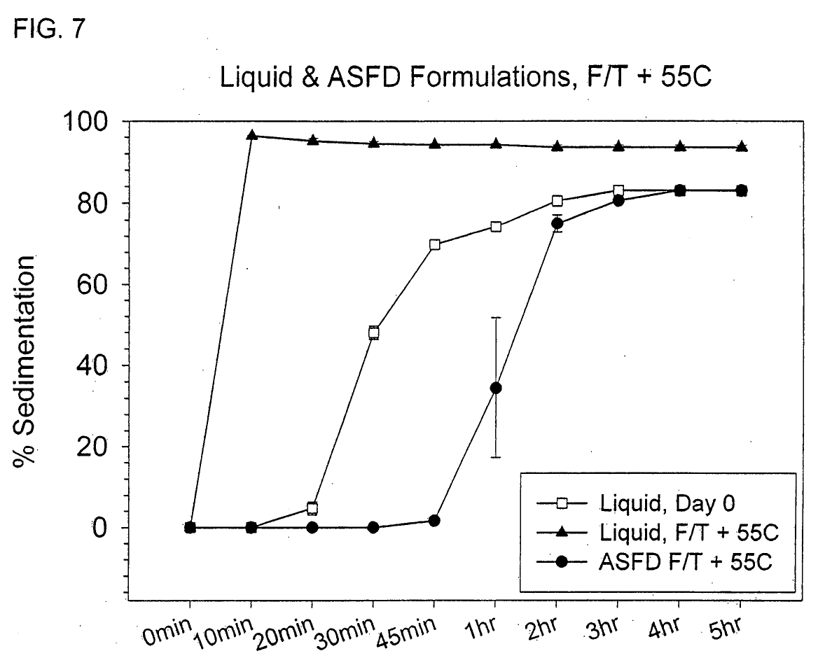

Sedimentation assays were performed as described in Example 2 using liquid Shanvac-B vaccine and reconstituted ASFD Shanvac-B vaccine formulations. The results obtained in the sedimentation assays are provided in FIGS. 5-7. No change in sedimentation rate was observed in either the liquid or ASFD formulations after storage at 4° C. for one month (FIG. 5). Following storage at 55° C., no increase in sedimentation rate (i.e., leftward shift in the sedimentation curve) resulting from agglomeration was observed in either the liquid or ASFD formulations (FIG. 6). In fact, the sedimentation curve obtained with the ASFD formulation displayed a rightward shift, suggesting a slower sedimentation rate following storage at 55° C. After freeze-thaw and storage at 55° C. for 14 days, the liquid formulation showed a substantially faster sedimentation rate (FIG. 7). Agglomerates within the formulation were clearly visible. No agglomeration, however, was observed in the ASFD processed vaccine under the same freeze-thaw and storage conditions, as indicated by the lack of change in the sedimentation rate. Moreover, visual analysis of the particles by optical microscopy indicated that the ASFD vaccine retained the small, non-aggregated aluminum hydroxide particle structure. Therefore, ASFD processing appears to protect the alum-adsorbed HBsAg vaccine from particle agglomeration.

A summary of the sedimentation data presented in FIGS. 5 and 6 is provided below in Tables 11-15.

TABLE 11
Summary of Sedimentation Data for Liquid Shanvac-B Vaccine
Formulations Following Storage at 0° C. for 0 and 28 Days

% Sedimentation

| Time | | Day 0 | Day 28 |
|---|---|---|---|
| 0 | min | 0 | 0 |
| 10 | min | 0.00 | 0.00 |
| 20 | min | 17.69 | 13.06 |
| 30 | min | 55.55 | 59.63 |
| 1 | hr | 73.77 | 75.77 |
| 2 | hr | 79.01 | 81.37 |
| 3 | hr | 81.22 | 82.61 |
| 4 | hr | 81.76 | 83.23 |
| 5 | hr | 81.76 | 83.23 |

TABLE 12
Summary of Sedimentation Data for ASFD Shanvac-B Vaccine
Formulations Following Storage at 0° C. for 0 and 28 Days

% Sedimentation

| Time | | Day 0 | Day 28 |
|---|---|---|---|
| 0 | min | 0.00 | 0.00 |
| 10 | min | 0.00 | 0.00 |
| 20 | min | 5.74 | 0.00 |
| 30 | min | 40.10 | 0.00 |
| 45 | min | 67.99 | 13.64 |
| 1 | hr | 72.75 | 66.55 |
| 2 | hr | 78.82 | 79.94 |
| 3 | hr | 79.42 | 82.98 |
| 4 | hr | 80.63 | 84.20 |
| 5 | hr | 81.52 | 85.41 |

TABLE 13
Summary of Sedimentation Data for Liquid Shanvac-B Vaccine
Formulations Following Storage at 55° C. for 0, 7, 14, and 28 Days

% Sedimentation

| Time | | Day 0 | Day 7 | Day 14 | Day 28 |
|---|---|---|---|---|---|
| 0 | min | 0 | 0 | 0 | 0 |
| 10 | min | 0.00 | 0.00 | 0.00 | 0.00 |
| 20 | min | 17.69 | 12.96 | 9.09 | 17.53 |
| 30 | min | 55.55 | 44.44 | 27.91 | 48.18 |
| 45 | min | 67.99 | 69.14 | 68.02 | 71.26 |
| 1 | hr | 73.77 | 75.31 | 75.55 | 75.62 |
| 2 | hr | 79.01 | 82.10 | 82.44 | 82.49 |
| 3 | hr | 81.22 | 83.33 | 84.32 | 83.75 |
| 4 | hr | 81.76 | 85.19 | 84.95 | 84.37 |
| 5 | hr | 81.76 | 85.19 | 84.95 | 84.37 |

TABLE 14
Summary of Sedimentation Data for ASFD Shanvac-B Vaccine
Formulations Following Storage at 55° C. for 0, 7, 14, and 28 Days

% Sedimentation

| Time | | Day 0 | Day 7 | Day 14 | Day 28 |
|---|---|---|---|---|---|
| 0 | min | 0 | 0 | 0 | 0 |
| 10 | min | 0.00 | 0.00 | 0.00 | 0.00 |
| 20 | min | 5.74 | 0.00 | 0.00 | 0.00 |
| 30 | min | 40.10 | 0.00 | 0.00 | 0.00 |
| 45 | min | 67.99 | 1.21 | 1.52 | 3.73 |
| 1 | hr | 72.75 | 15.76 | 5.45 | 31.53 |
| 2 | hr | 78.82 | 73.33 | 73.33 | 76.21 |
| 3 | hr | 79.42 | 80.61 | 78.18 | 81.24 |
| 4 | hr | 80.63 | 82.42 | 80.00 | 83.12 |
| 5 | hr | 81.52 | 84.24 | 82.42 | 83.12 |

TABLE 15
Summary of Sedimentation Data for Liquid and ASFD Shanvac-B Vaccine
Formulations Following a Freeze-Thaw Cycle and Storage at 55° C.
for 14 Days

| | | % Sedimentation | | |
|---|---|---|---|---|
| Time | | Liquid d0 | Liquid F/T+55 C | ASED F/T+55 C |
| 0 | min | 0 | 0 | 0 |
| 10 | min | 0.00 | 96.32 | 0.00 |
| 20 | min | 17.69 | 95.10 | 0.00 |
| 30 | min | 55.55 | 94.48 | 0.00 |
| 45 | min | 67.99 | 94.18 | 1.57 |
| 1 | hr | 73.77 | 94.18 | 34.45 |
| 2 | hr | 79.01 | 93.56 | 75.05 |
| 3 | hr | 81.22 | 93.56 | 80.62 |
| 4 | hr | 81.76 | 93.56 | 83.11 |
| 5 | hr | 81.76 | 93.56 | 83.11 |

### AUSZYME Assay

AUSZYME assays were performed as described in Example 2 using liquid Shanvac-B vaccine and reconstituted ASFD Shanvac-B formulations. The AUSYME assay measures the concentration of HBsAg by an ELISA-based method. This assay uses antibodies against HBsAg as a means of capturing the antigen for determination of its concentration by comparison with standard reference curves. The ability of the antibodies to bind to HBsAg in this assay makes it suitable for a measure of immunogen concentration and of the antigenicity of the HBsAg. As described herein above, “antigenicity” is defined as the ability of an antibody to recognize and bind to a protein (i.e., an immunogen). In comparison, “immunogenicity” refers to the ability of the protein (i.e., immunogen) to raise an immune response in vivo (e.g., in a human or non-human subject). The results of the AUSZYME assay are presented in Table 16 below.

TABLE 16
Concentration of HBsAg in Liquid and ASFD Hepatitis B Vaccine
Formulations Under Various Storage Conditions

| | | | | Freeze-thaw + | | |
|---|---|---|---|---|---|---|
| | Day 0 | Day 7, 55° C. | Day 14, 55° C. | Day 14, 55° C. | Day 28, 55° C. | Day 28, 4° C. |
| Liquid | 10 | 7.08 | 2.96 | 7.69 | 1 | 11.75 |
| ASFD M/T | 7.8 | 9.19 | 7.23 | 7.38 | 8.19 | 9.57 |

*M/T = mannitol/trehalose

The AUSZYME assay results demonstrate that the liquid and ASFD Shanvac-B vaccine formulations retained their original HBsAg concentration ([HBsAg]) when stored at 4° C. for 28 days. When the liquid Shanvac vaccine was stored at 55° C., however, the [HBsAg] began to decrease following 7 days and then dropped significantly following 14 and 28 days of storage. In contrast, the [HBsAg] observed with the ASFD processed vaccine did not decrease when stored at 55° C. for 28 days. Therefore, the ASFD process prevented a decrease in [HBsAg] resulting from storage at high temperatures.

As noted above in the analysis of the SFD processed vaccine formulation, neither the ASFD nor the liquid Shanvac-B formulation exhibited a decrease in [HBsAg] when freeze-thawed and then stored at 55° C. for 14 days. This is surprising considering that the liquid formulation exhibited agglomeration at this storage condition as measured by sedimentation assays and that the [HBsAg] of the liquid vaccine dropped markedly following storage at 55° C. over 28 days. It is speculated that agglomerates formed in the liquid formulation during the freeze-thaw cycle, thereby encapsulating and helping to protect the HBsAg against degradation during the subsequent storage at 55° C.

### Immunogenicity Studies

To evaluate whether the ASFD hepatitis B vaccine formulation retained immunogenicity, mice were immunized with various formulations of the liquid or reconstituted ASFD or SFD Shanvac-B vaccine. The immune response was measured by quantifying the serum HBsAg-specific antibody response, as detailed above in Example 2. Specifically, female Balb/c mice (10 per group), were immunized at day 0 and at day 28 by intramuscular injection with a 2 microgram dose of one of the following vaccine formulations:

1. Shanvac-B vaccine (liquid)
2. Shanvac-B vaccine+mannitol/trehalose (liquid)
3. ASFD Shanvac-B+mannitol/trehalose (reconstituted)
4. SFD Shanvac-B+mannitol/trehalose (reconstituted)

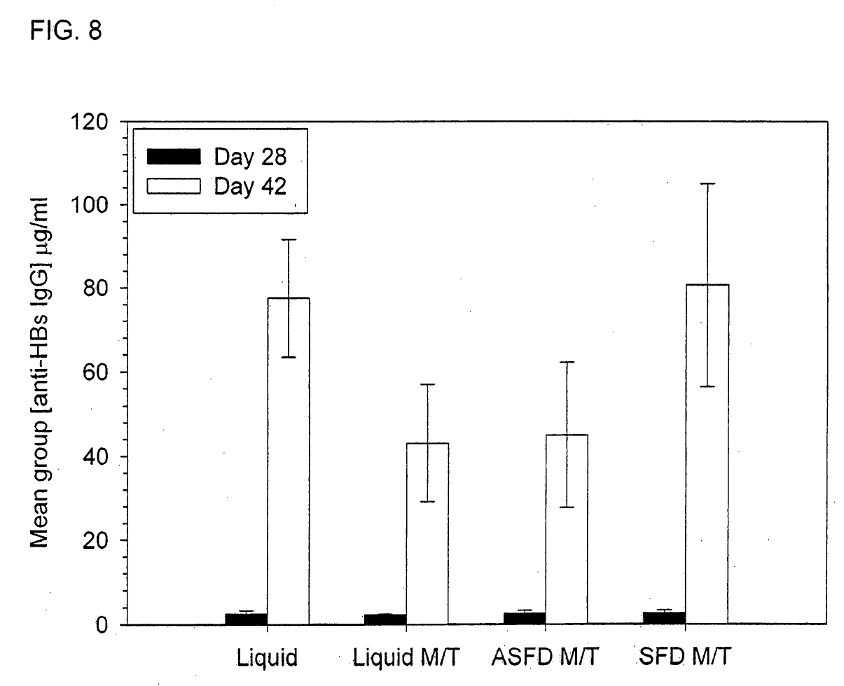

The results of the ASFD vaccine immunogenicity studies are summarized in FIG. 8 and in Tables 17 and 18 below. Immunization of mice with reconstituted ASFD vaccine powder formulations resulted in antibody production at levels similar to those obtained following immunization with conventional liquid vaccine (FIG. 8). After the second immunization, the antibody levels generated by the ASFD formulation were not significantly different than those obtained following immunization with either the liquid or SFD formulations (p>0.05). These results demonstrate that the ASFD process does not negatively affect the immunogenicity of the Shanvac-B vaccine.

TABLE 17
Summary of Mean Anti-HBsAg Antibody Concentrations (μg/ml) in
Serum from Mice Immunized with Liquid or SFD Hepatitis B Vaccine
Formulations (28 Days Post-Immunization)

Day 28
Dose of HBsAg
2ug
Formulation

| | | |
|---|---|---|
| Liquid | ASFD | SFD |

Excipient

| | | | |
|---|---|---|---|
| N/A | T/M | T/M | T/M |

Group #

| | 1 | 2 | 3 | 4 |
|---|---|---|---|---|
| 1 | 1.73 | 0.71 | 3.78 | 2.84 |
| 2 | 4.62 | 1.26 | 5.28 | 2.37 |
| 3 | 2.49 | 1.34 | 1.46 | 1.10 |
| 4 | 2.22 | 0.70 | 0.01 | 1.87 |
| 5 | 1.74 | 0.01 | 2.93 | 2.82 |
| 6 | 3.12 | 1.09 | 0.01 | 1.66 |
| 7 | 7.76 | 2.16 | 3.12 | 3.16 |
| 8 | 0.82 | 0.01 | 3.89 | 2.58 |
| 9 | 0.01 | 0.87 | 1.44 | 8.39 |
| 10 | 0.67 | 1.45 | 6.89 | 1.65 |
| Group Mean | 2.52 | 2.31 | 2.58 | 2.67 |

TABLE 18
Summary of Mean Anti-HBsAg Antibody Concentrations (μg/ml) in
Serum from Mice Immunized with Liquid or SFD Hepatitis B Vaccine
Formulations (42 Days Post-Immunization)

Day 42
Dose of HBsAg
2ug
Formulation

| | | |
|---|---|---|
| Liquid | ASFD | SFD |

Excipient

| | | | |
|---|---|---|---|
| N/A | T/M | T/M | T/M |

Group #

| | 1 | 2 | 3 | 4 |
|---|---|---|---|---|
| 1 | 163.11 | 139.68 | 21.83 | 54.25 |
| 2 | 22.32 | 29.66 | 100.36 | 20.03 |
| 3 | 77.49 | 31.17 | 17.90 | 30.98 |
| 4 | 37.16 | 98.16 | 15.75 | 59.90 |
| 5 | 116.24 | 15.37 | 180.89 | 49.46 |
| 6 | 116.75 | 15.14 | 39.32 | 8.97 |
| 7 | 88.51 | 61.95 | 30.11 | 142.69 |
| 8 | 40.93 | 2.38 | 7.23 | 30.83 |
| 9 | 40.66 | 21.11 | 23.33 | 169.73 |
| 10 | 72.30 | 15.56 | 12.59 | 240.54 |
| Group Mean | 77.55 | 43.02 | 44.93 | 80.74 |

### Example 4

### Analysis of SFD BoNT/A Alum-Adsorbed Vaccine

### Preparation of SFD Bont/a Alum-Adsorbed Vaccine

A recombinant BoNT/A HC immunogen (provided by United States Army Medical Research Institute of Infectious Disease; Fort Detrick, Md.) was adsorbed onto either aluminum hydrogel (i.e., aluminum hydroxide) for intramuscular (IM) or intradermal (ID) injection or adsorbed onto lipopolysaccharide (LPS) for intranasal (IN) delivery. BoNT/A vaccines were formulated as traditional liquid vaccines or as dry powder vaccine formulations by SFD, in accordance with the methods described herein. See generally Example 1. The SFD BoNT/A vaccine powder formulations were reconstituted with water immediately prior to administration to a subject.

Immunizations and Challenge with BoNT/A

6-8 week old female CD-1/ICR mice (Charles River) were employed for analysis of the liquid and reconstituted powder BoNT/A vaccine formulations. Specifically, 10 mice per test group received the liquid or reconstituted BoNT powder vaccine formulation as an IM injection or a microneedle-based ID injection at a volume of 50 μl (25 μl per side) or, alternatively, by IN administration of 30 μl of the formulation (15 μl per nostril) at days 0 and 28. As negative controls, specific groups of mice were either left un-immunized or were given liquid formulations containing adjuvant only by IM or IN administration. Blood was collected on days 0, 14, 28 and 42, in accordance with standard techniques in the art. All mice were challenged with a lethal dose of BoNT/A (100,000×the mouse LD₅₀ of BoNT/A) on day 49 and then observed for an additional five days. Survival rates of mice from the various test groups were assessed at day 54 and are presented in Table 19 below.

TABLE 19
Percent Survival Following Lethal Challenge with BoNT/A

| | | BoNT | |
|---|---|---|---|
| Delivery Route | Formulation | vaccine dose (μg) | % Survival |
| IM | Liquid | 1.0 | 100 |
| IM | SFD | 1.0 | 100 |
| IM | Liquid | 0.1 | 100 |
| IM | SFD | 0.1 | 80 |
| ID | Liquid | 1.0 | 100 |
| ID | SFD | 1.0 | 100 |
| ID | Liquid | 0.1 | 90 |
| ID | SFD | 0.1 | 100 |
| IN | Liquid | 1.0 | 40 |
| IN | SFD | 1.0 | 50 |
| IN | Liquid | 0.1 | 10 |
| IN | SFD | 0.1 | 10 |
| IM/IN | Liquid (adjuvant only) | — | 0 |
| Unimmunized | — | — | 0 |

IM and ID immunization with the reconstituted SFD BoNT/A powder vaccine produced survival rates (up to ˜100%) similar to those observed with the liquid BoNT/A vaccine formulation. IN delivery of either the reconstituted SFD or liquid BoNT/A vaccine resulted in similar survival rates (up to ˜50%) that were lower than those observed with either IM or ID immunization with the same BoNT/A vaccines.

### Antibody Serum Titer Analysis and Statistical Analyses

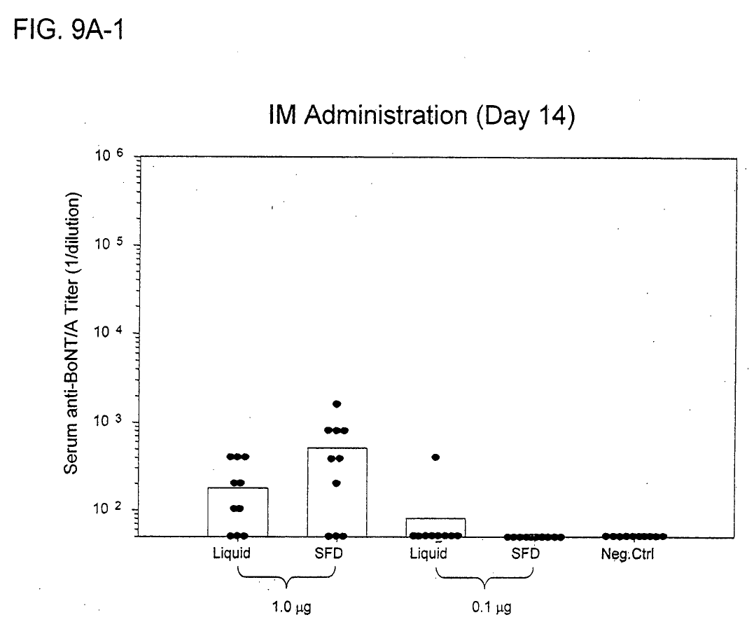

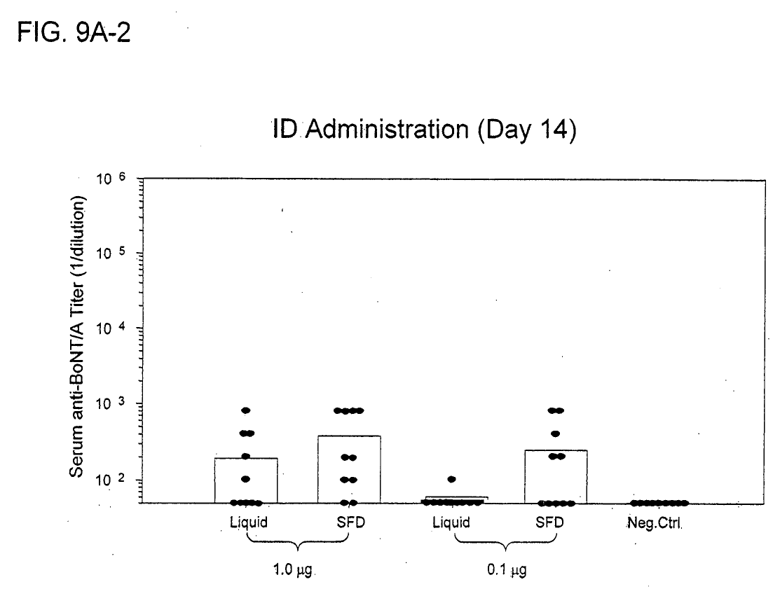

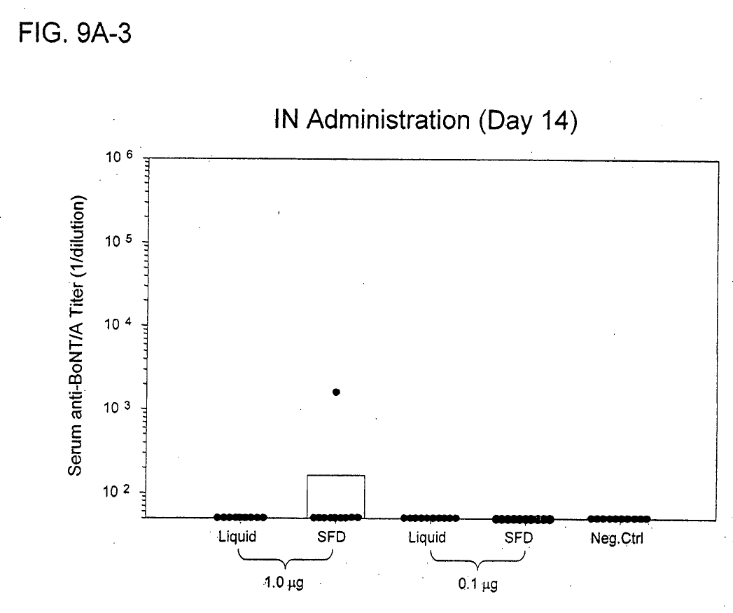

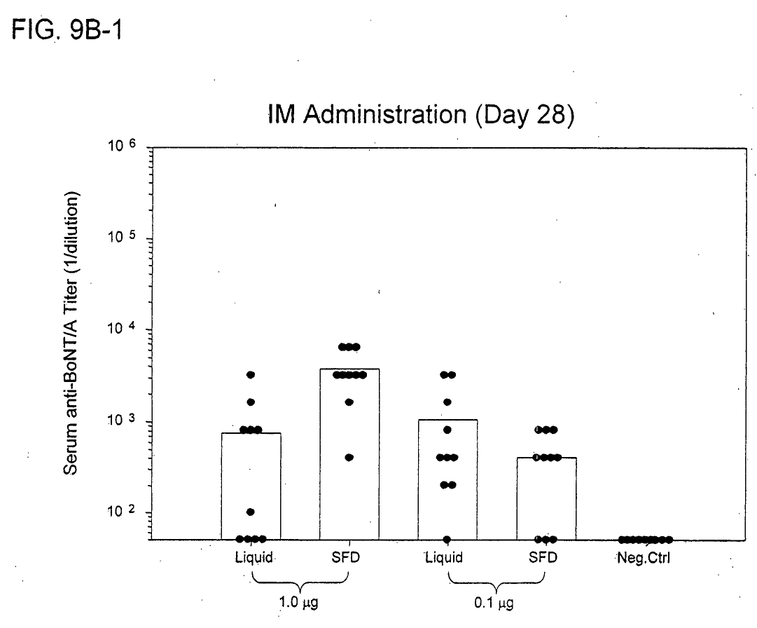

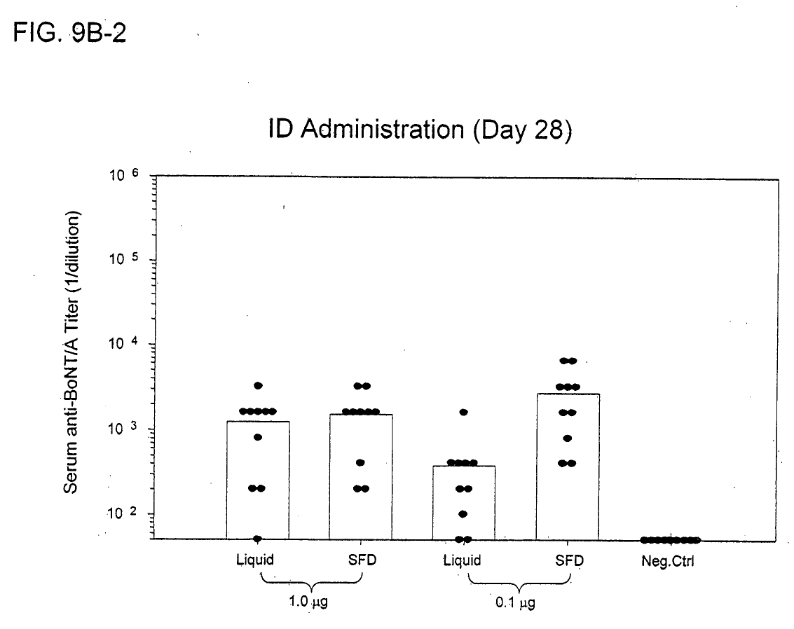

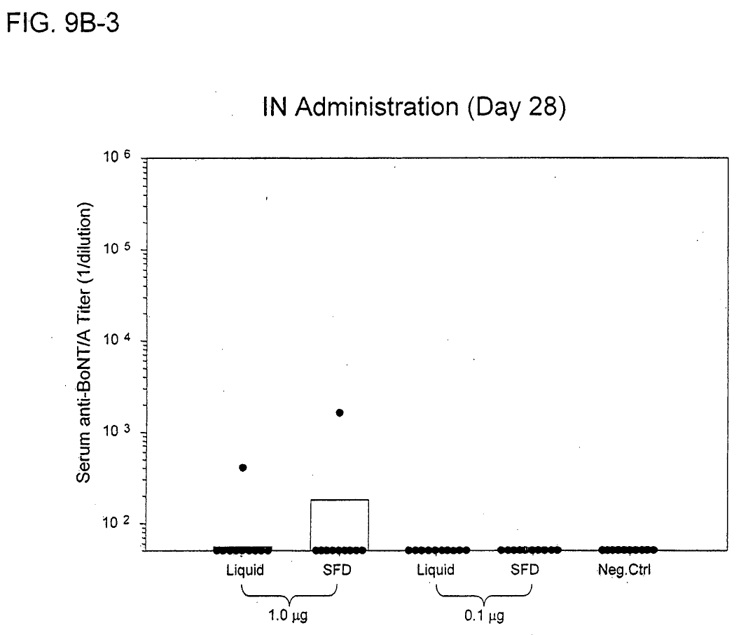

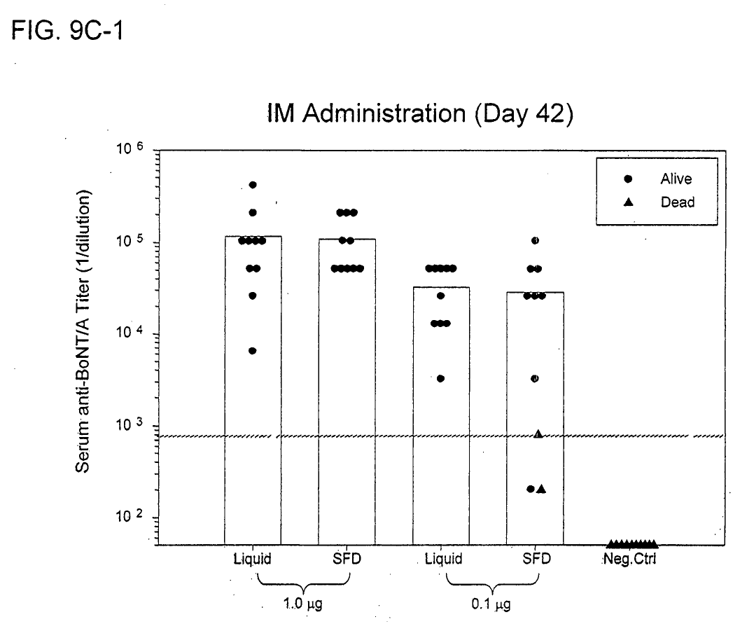

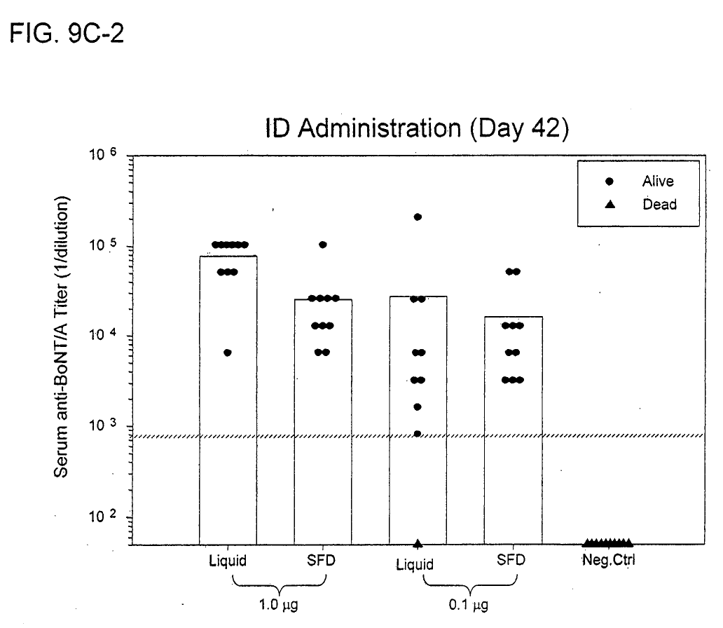

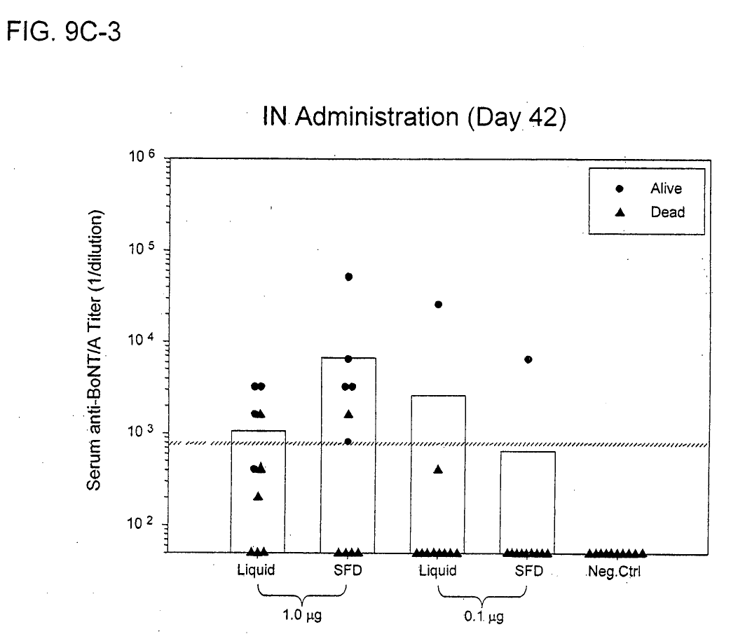

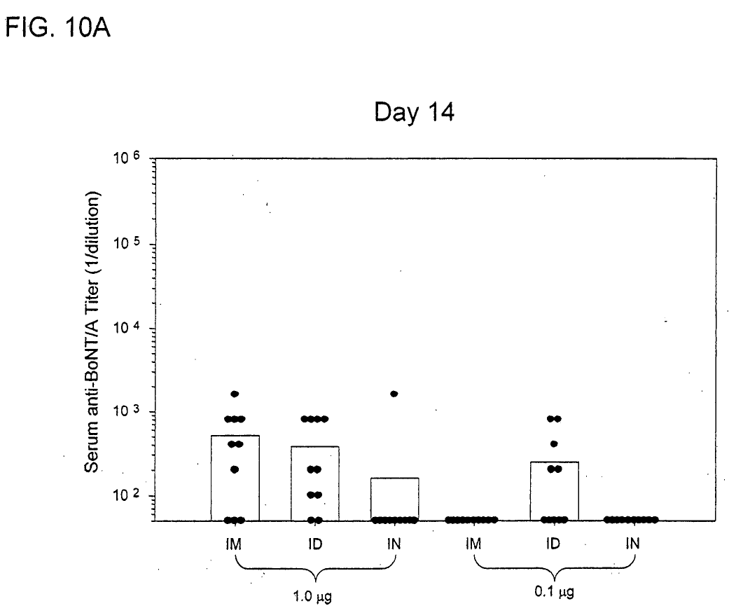

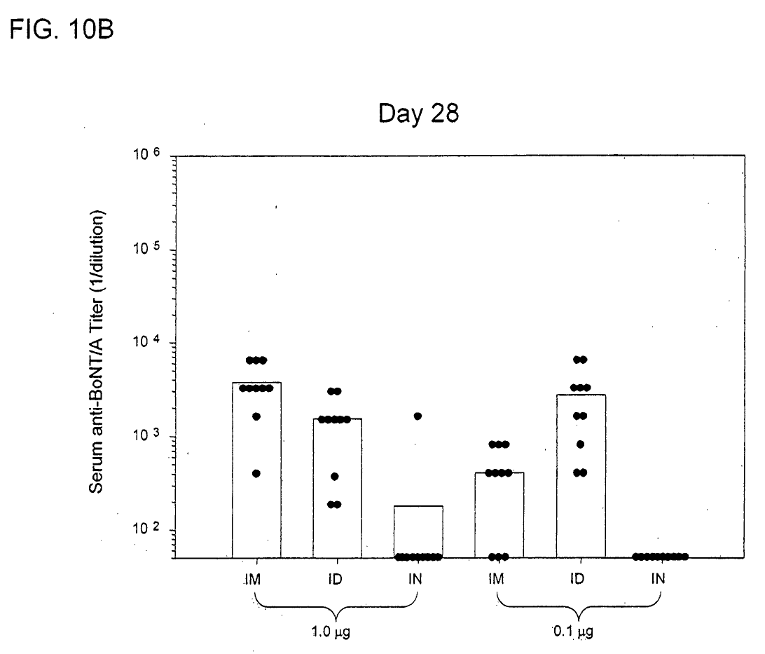

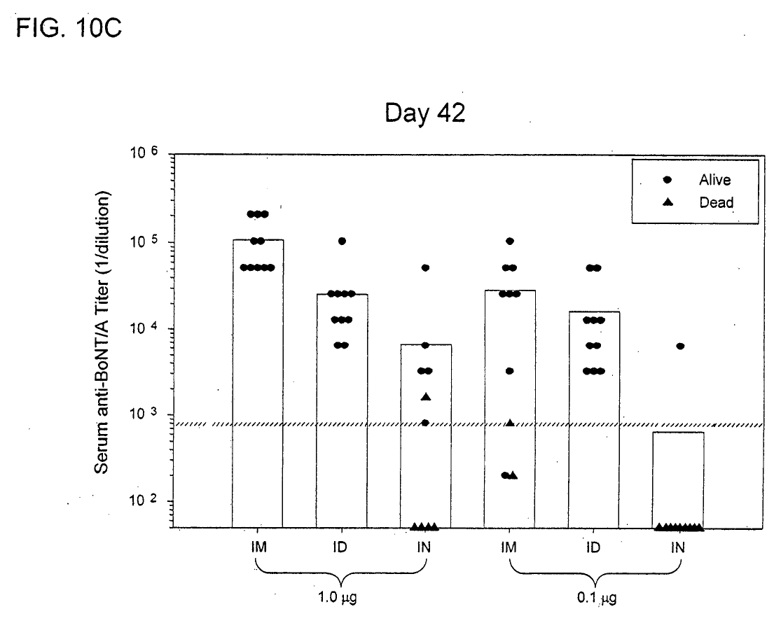

Blood serum samples from subjects from the various test groups were analyzed for BoNT/A-specific IgG titers by standard ELISA techniques. Results are summarized in FIGS. 9 and 10. Statistical analyses of serum antibody titers and survival rates observed with lethal challenge with BoNT/A were performed by ANOVA and Receiver Operating Characteristic (ROC) curve analysis. An antibody titer of 800, at which subjects have a 97% rate of survival from lethal challenge with BoNT/A is indicated by a dotted line in FIGS. 9C and 10C.

Both IM and ID immunization with the reconstituted SFD BoNT/A powder vaccine produced a strong antibody response, with mean serum BoNT/A antibody levels similar to those observed with the liquid BoNT/A vaccine formulation. IN delivery of either the reconstituted SFD or liquid BoNT/A vaccine produced a lower antibody response than that observed with either IM or ID immunization with the same BoNT/A vaccine formulations.

### Example 5

### Analysis of SFD and ASFD B. anthracis rPA Alum-Adsorbed Vaccine

Preparation of SFD and ASFD B. anthracis rPA Alum-Adsorbed Vaccine

Dried powder formulations of B. anthracis rPA vaccines were prepared by either SFD or ASFD (with liquid or gaseous nitrogen processing), as described below, and reconstituted in a pharmaceutically acceptable carrier prior to immunization of mice. The efficacy of the reconstituted SFD and ASFD B. anthracis rPA vaccines was then assessed by quantifying the anthrax lethal toxin neutralization antibody titer in the sera of immunized animals, as outlined in detail below.

### Spray-Freeze-Drying (SFD)

Powder formulations of B. anthracis rPA vaccine were produced by SFD essentially as described in Example 1 above and in Jiang et al. (2006) J. Pharm. Sci. 95:80-96. Briefly, a liquid formulation comprising B. anthracis rPA, Alhydrogel®, Tween® 80, and mannitol/trehalose was prepared and sprayed using a BD Accuspray® nozzle affixed to a 5 ml syringe. 5 ml aliquots were sprayed into a metal pan containing liquid nitrogen, and the pan was transferred to a shelf lyophilizer pre-cooled to −40 C. The liquid nitrogen was allowed to completely evaporate, and the powder was then dried as described in Table 1 above.

The powder vaccine samples were removed from the lyophilizer, placed in a dry glove box (5% relative humidity (“RH”)), and then transferred into glass containers and sealed. The final SFD vaccine powder contained 0.5% B. anthracis rPA and 1% aluminum.

### Atmospheric Spray-Freeze-Drying (ASFD)—Liquid Nitrogen (LN₂) Processing

Powder formulations of B. anthracis rPA vaccine were produced by ASFD essentially as described in U.S. Patent Application Publication No. 2003/0180755. Specifically, a liquid formulation comprising B. anthracis rPA, Alhydrogel®, Tween® 80, and mannitol/trehalose was prepared. A syringe was charged with this formulation and sprayed using a BD Accuspray® nozzle affixed to a 5 ml syringe into an ASFD chamber filled with liquid nitrogen. When the spraying was completed, a dry nitrogen gas flow was initiated into the bottom of the chamber at a rate of 40 L/min. The dry nitrogen gas flow continued until all of the liquid nitrogen evaporated. The inlet gas flow temperature was then increased to obtain an outlet gas temperature of −35° C. with the gas flow rate set at 140 L/min to anneal the powder for one hour prior to drying. The gas flow was then reduced to 60 L/min and warmed to obtain to the desired primary drying temperature of −20° C. at the outlet. After 24 hours when the measured percent RH dropped to less than 0.015%, the gas temperature was again warmed to obtain an outlet temperature of 0° C. to proceed with secondary drying, keeping the gas flow at 60 L/min. After 7 hours when the measured percent RH dropped to less than 0.015%, the inlet gas was warmed to obtain an outlet temperature of 23° C., at which point the gas flow was shut off and the powder harvested. The final ASFD vaccine powder contained 0.5% B. anthracis rPA and 1% aluminum.

### Atmospheric Spray-Freeze-Drying (ASFD)—Gaseous Nitrogen (GN₂) Processing

Powder formulations of B. anthracis rPA vaccine were produced by ASFD essentially as described in U.S. Patent Application Publication No. 2003/0180755. Specifically, a liquid formulation comprising B. anthracis rPA, Alhydrogel®, Tween® 80, and mannitol/trehalose was prepared. A syringe was charged with this formulation and sprayed using a Sono-Tek Corporation Model 8700-25 ultrasonic nozzle into an ASFD chamber pre-cooled to −80° C. with gaseous nitrogen at a flow rate of 40 L/min. When the spraying was completed, the inlet gas flow temperature was increased to obtain an outlet gas temperature of −35° C. with the gas flow rate set at 140 L/min to anneal the powder for one hour prior to drying. The gas flow was then reduced to 60 L/min and warmed to obtain the desired primary drying temperature of −20° C. at the outlet. After 24 hours when the measured percent RH dropped to less than 0.015%, the gas temperature was again warmed to obtain an outlet temperature of 0° C. to proceed with secondary drying, while maintaining the gas flow rate at 60 L/min. After 3 hours when the measured percent RH dropped to less than 0.015%, the inlet gas was warmed to obtain an outlet temperature of 23° C., at which point the gas flow was shut off and the powder harvested. The final ASFD vaccine powder contained 0.5% B. anthracis rPA and 1% aluminum.

### Immunizations

Mice were immunized with various formulations of the liquid or reconstituted SFD or ASFD B. anthracis rPA vaccines. The mice in each test group were immunized at day 0 and at day 28 by intramuscular injection with one of the vaccine formulations listed in Table 20 or Table 21. Mice were bled at day 0 (bleed #1 or “pre-bleed”), day 14 (bleed #2 or “b2”), day 28 (bleed #3 or “b3”), and day 42 (bleed #4 or “b4”). Serum samples were then analyzed using the anthrax lethal toxin neutralization assay described below.

TABLE 20
B. anthracis rPA Vaccine Test Groups (Set 1)

| Group | Process | Nozzle |
|---|---|---|
| 1 | Liquid | N/A |
| 2 | Liquid | Accuspray ® |
| 3 | ASFD-LN₂ | Accuspray ® |
| 4 | ASFD-LN₂ | Ultrasonic |
| 5 | SFD | Accuspray ® |

*“Liquid” refers to a B. anthracis rPA vaccine formulation that has not been subjected to SFD or ASFD; LN₂ indicates that the ASFD process was performed with liquid nitrogen processing, as described above.

TABLE 21
B. anthracis rPA Vaccine Test Groups (Set 2)

| Group | Process | Nozzle |
|---|---|---|
| 1 | Liquid | N/A |
| 2 | SFD | Accuspray |
| 3 | SFD | Ultrasonic |
| 4 | ASFD-LN₂ | Ultrasonic |
| 5 | ASFD-GN₂ | Ultrasonic |

*“Liquid” refers to a B. anthracis rPA vaccine formulation that has not been subjected to SFD or ASFD; LN₂ indicates that the ASFD process was performed with liquid nitrogen processing; GN₂ indicates that the ASFD process was performed with gaseous nitrogen processing, as described above.

### Anthrax Lethal Toxin Neutralization Assay

Anthrax lethal toxin neutralizing antibody titers in the sera of mice from the various test groups were determined. Anthrax lethal toxin neutralization assays are known in the art. See, for example, Little et al. (1990) Infect. Immunol. 58(6):1606-1613 and Hering et al. (2004) Biologicals 32(1):17-27. In the present example, dilutions of serum samples were mixed with B. anthracis rPA and the anthrax toxin lethal factor. The mixtures were incubated and added to cell monolayers. The anthrax toxin-serum mixture was then incubated with the cells. Cell viability in the presence of the anthrax toxin-serum mixture was assessed by staining the cells and by measuring the optical density. Neutralizing antibody titers represented the highest serum dilution at which the anthrax toxin was neutralized.

### Results

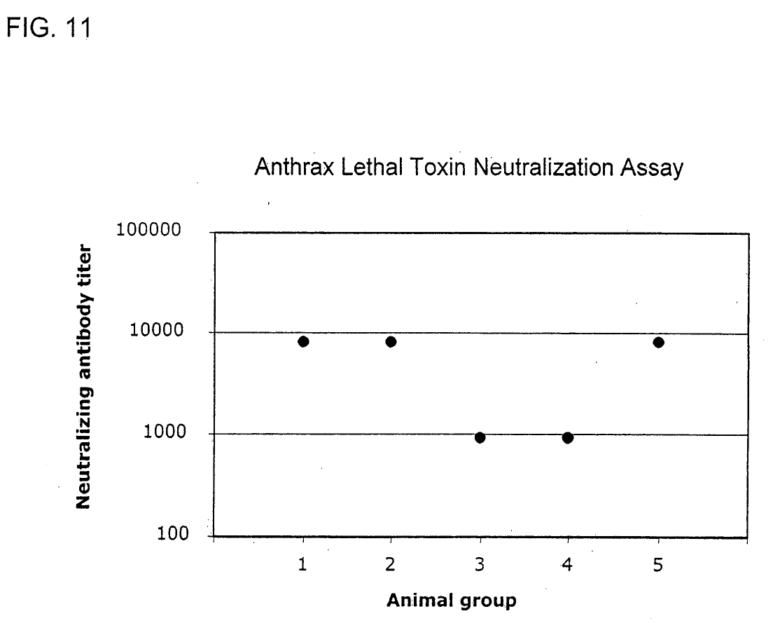

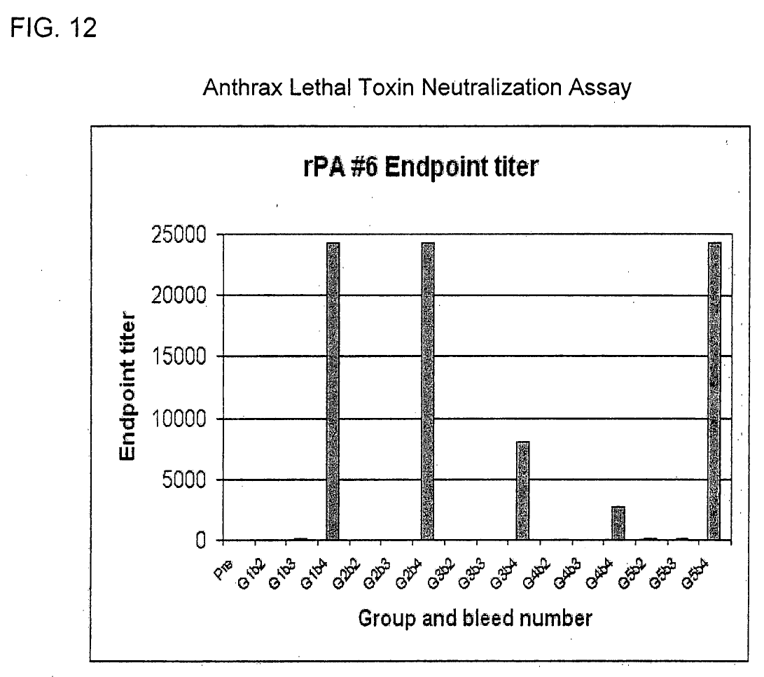

The results from the anthrax lethal toxin neutralization assays obtained with the B. anthracis rPA vaccine formulations of sets 1 and 2 (see Tables 20 and 21 above) are presented in FIGS. 11 and 12, respectively. The data shown in FIG. 11 demonstrate that the SFD powder produced with the Accuspray® nozzle (Set 1, Group 4) retains the ability to induce toxin neutralizing antibodies at a level equivalent to that obtained with the unprocessed liquid vaccine (Group 1; Set 1). The data shown in FIG. 12 further demonstrate that the SFD powder produced with the Accuspray® nozzle (Set 2, Group 4) and the ASFD powder produced by the GN₂ process (Set 2; Group 5) both retain the ability to induce toxin neutralizing antibodies at a level equivalent to that obtained with the unprocessed liquid vaccine.

### Example 6

### Analysis of SFD Y. pestis Alum-Adsorbed Vaccine Formulations

Preparation of SFD Y. pestis F1-V Alum-Adsorbed Vaccine Formulations

F1-V protein was provided by the National Institutes of Allergy and Infectious Diseases and formulated with various excipients and adjuvants as a liquid suspension (i.e., without SFD processing) or as a dried powder processed by SFD, essentially as described herein above in Example 1. Pressure diafiltration was used to prepare 4.5 ml of F1-V solution in a buffer containing 20 mM Tris, 50 mM MgCl₂ and 2% Tween® 80 (pH 7.4). The concentration of F1-V in this buffer was 0.609 mg/ml.

The F1-V immunogen was adsorbed onto Alhydrogel® prior to the SFD process, and formulations of the Y. pestis alum-adsorbed vaccine were prepared, as described below. The SFD vaccine powder formulations were reconstituted in water for injection prior to immunization of the mice.

Group 1: In a 2-ml vial 0.133 ml of F1-V protein (at a concentration of 1.5 mg/ml), 0.1 ml of Alhydrogel®, and 0.767 ml of buffer (i.e., 10 mM NaCl, 20 mM arginine, and 1 mM cystine at pH 9.9) were mixed until a uniform suspension was formed.
Groups 2 and 3: In a 10-ml vial 114.2 mg of mannitol, 12.6 mg of trehalose, 0.466 ml of F1-V, 0.35 ml Alhydrogel®, and 2.685 ml of buffer (i.e., 10 mM NaCl, 20 mM arginine, and 1 mM cystine at pH 9.9) were mixed until a uniform suspension was formed. A 1.0 ml aliquot was removed and used as-is in liquid form (i.e., Group 2). The remainder of the suspension was sprayed into liquid nitrogen using an Accuspray® nozzle attached to a 1-ml syringe. This frozen sample was placed in a shelf lyophilizer pre-cooled to −45° C. and dried under vacuum (i.e., Group 3).
Group 4: In a 2-ml vial 0.284 ml of buffer exchanged F1-V (at a concentration of 0.703 mg/ml), 0.1 ml Alhydrogel®, and 0.616 ml of buffer (i.e., 10 mM NaCl, 20 mM arginine, 1 mM cystine, pH 9.9) were mixed until a uniform suspension was formed.
Groups 5 and 6: In a 10-ml vial 125.8 mg of mannitol, 13.9 mg of trehalose, 0.994 ml of F1-V, 0.35 ml of Alhydrogel®, and 2.156 ml of buffer (10 mM NaCl, 20 mM arginine, 1 mM cystine, pH 9.9) were mixed until a uniform suspension was formed. A 1.0 ml aliquot was removed and used as-is in liquid form (i.e., Group 5). The remainder of the suspension was sprayed into liquid nitrogen using an Accuspray® nozzle attached to a 1-ml syringe. This frozen sample was placed in a shelf lyophilizer pre-cooled to −45° C. and dried under vacuum (i.e., Group 6).

TABLE 22
Summary of Y. pestis F1-V Vaccine Test Groups

| Group | Dose (μg) | Formulation | Processing |
|---|---|---|---|
| 1 | 10 | Al(OH)₃ | Liquid |
| | | | Suspension |
| 2 | 10 | Al(OH)₃/Mann*/Tre+ | Liquid |
| | | | Suspension |
| 3 | 10 | Al(OH)₃/Mann*/Tre+ | SFD |
| 4 | 3.3 | Al(OH)₃/Tween80/MgCl₂ | Liquid |
| | | | Suspension |
| 5 | 3.3 | Al(OH)₃/Mann/Tre+/Tween80/MgCl₂ | Liquid |
| | | | Suspension |
| 6 | 3.3 | Al(OH)₃/Mann/Tre+/Tween80/MgCl₂ | SFD |

### Immunizations

6-8 week old female Swiss Webster mice were housed and immunized with the above vaccine formulations prior to Y. pestis lethal challenge. Ten mice were used for each test group. Each mouse was immunized intramuscularly with 50 μl (25 μl per site) of the specified vaccine formulation at days 0 and 28 and at a dose of 3.3 μg or 10 μg of F1-V. Blood was collected on days 0, 14, 28 and 42 to assess antibody titers.

### Results

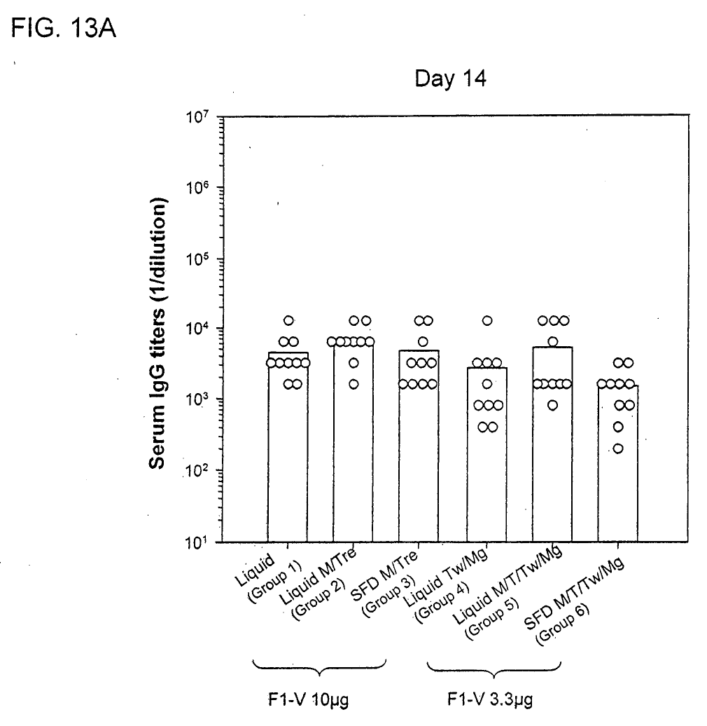

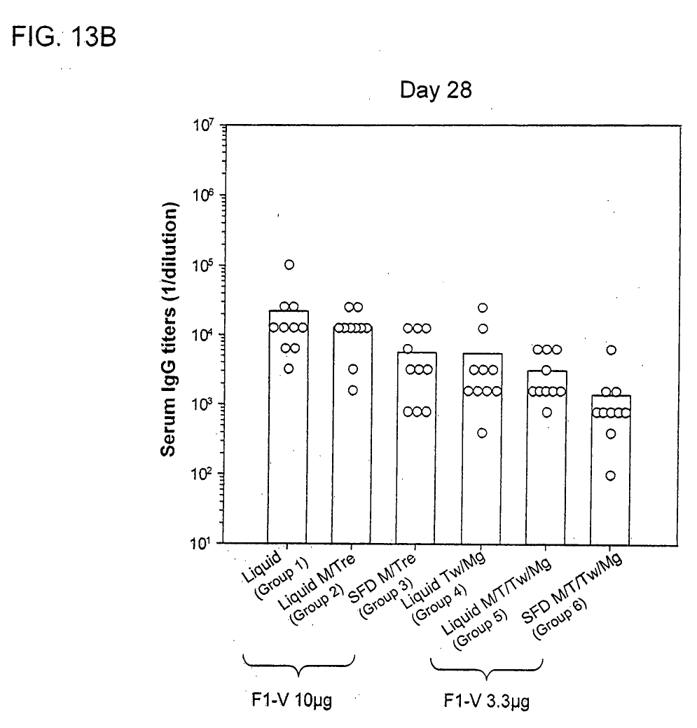

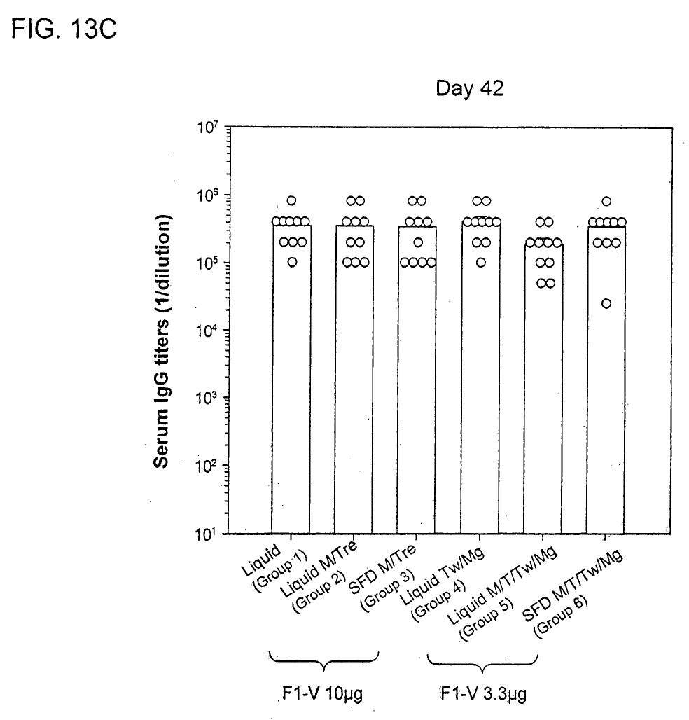

The results obtained with the Y. pestis F1-V vaccine formulations of Groups 1-6 are presented in FIG. 13. The data shown in this figure demonstrate that the reconstituted SFD F1-V powder vaccine resulted in antibody production at levels similar to those obtained following immunization with the liquid vaccine suspensions.

### Example 7

### Analysis of SFD Polyvalent Alum-Adsorbed Vaccine Formulations

### Preparation of SFD Polyvalent Alum-Adsorbed Vaccine Formulations

A polyvalent vaccine comprising four antigens (i.e., rPA, rSEB, BoNT/A, and F1-V) in a single formulation was prepared. In particular, solutions of F1-V (pH 9.0) were first processed by pressure diafiltration to exchange the buffer/excipients to 20 mM Tris, 50 mM MgCl₂, and 2% Tween® 80 (pH 7.4). Solutions of the other three antigens of interest (i.e., rPA, BoNT/A, and rSEB), were added to this solution to create the polyvalent solution. Alhydrogel® was added to this polyvalent solution, along with mannitol and trehalose excipients to produce an alum-adsorbed liquid suspension, and the suspension was then spray-freeze dried to produce a dried powder, essentially as described in Example 1. The resulting SFD powder was reconstituted in water for injection at the time of use.

The initial and final concentrations of each antigen are listed below:

Initial antigen concentration (as received from vendor):

rPA: 2.5 mg/mL rPA

rSEB: 3.4 mg/mL

BoNT/A: 0.18 mg/mL

F1-V: 600 μg/mL

Final antigen concentration (in dosed liquid and reconstituted SFD vaccine powder formulations):

rPA: 200 μg/mL

rSEB: 400 μg/mL

BoNT/A: 20 μg/mL

F1-V: 200 μg/mL

### Immunizations

Female BALB/c mice were immunized with a liquid or reconstituted SFD polyvalent vaccine formulation. Liquid monovalent vaccine formulations (i.e., comprising only one of the rPA, rSEB, BoNT/A, or F1-V antigens) were used as controls. Ten mice were used for each test group, and each mouse was immunized intramuscularly (IM) or intradermally (ID) with 50 μl (25 μl per site) of the specified vaccine formulation at days 0 and 28. The details of the test groups are summarized below in Table 23. Pre-bleed samples were collected from naïve, un-immunized mice. All test groups were bled on days 14, 28, and 42, and serum samples were analyzed by standard ELISA methods to determine antibody titers for each antigen (i.e., rPA, rSEB, BoNT-A and F1-V.)

TABLE 23
Summary of Polyvalent Vaccine Test Groups

| | | | rSEB | BoNT | F1-V | Alhydrogel | |
|---|---|---|---|---|---|---|---|
| | Method of | rPA Dose | Dose | Dose | Dose | Dose | |
| Group | Delivery | (μg) | (μg) | (μg) | (μg) | (% Al(OH)₃)* | Formulation |
| 1 | IM | 10 | 20 | 1 | 10 | 0.5 | Liquid |
| 2 | IM | 10 | 20 | 1 | 10 | 0.5 | Reconstituted |
| | | | | | | | SFD |
| 3 | IM | 10 | 0 | 0 | 0 | 0.5 | Liquid |
| 4 | IM | 0 | 20 | 0 | 0 | 0.5 | Liquid |
| 5 | IM | 0 | 0 | 1 | 0 | 0.5 | Liquid |
| 6 | IM | 0 | 0 | 0 | 10 | 0.5 | Liquid |
| 7 | IM | 0 | 0 | 0 | 0 | 0 | Liquid (naïve |
| | | | | | | | control) |
| 8 | IM | 10 | 20 | 1 | 10 | 0.25 | Liquid |
| 9 | ID | 10 | 20 | 1 | 10 | 0.25 | Liquid |

*Calculation based on amount in the final suspension for injection.

### Results

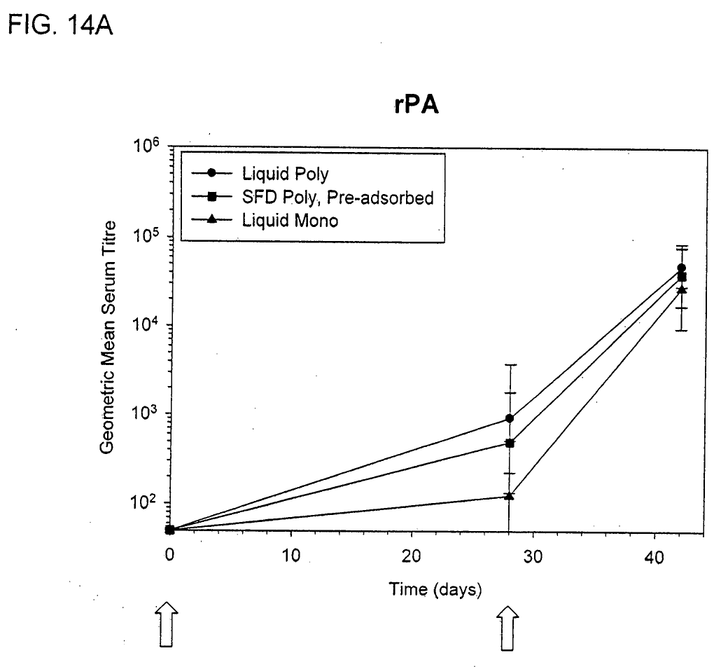

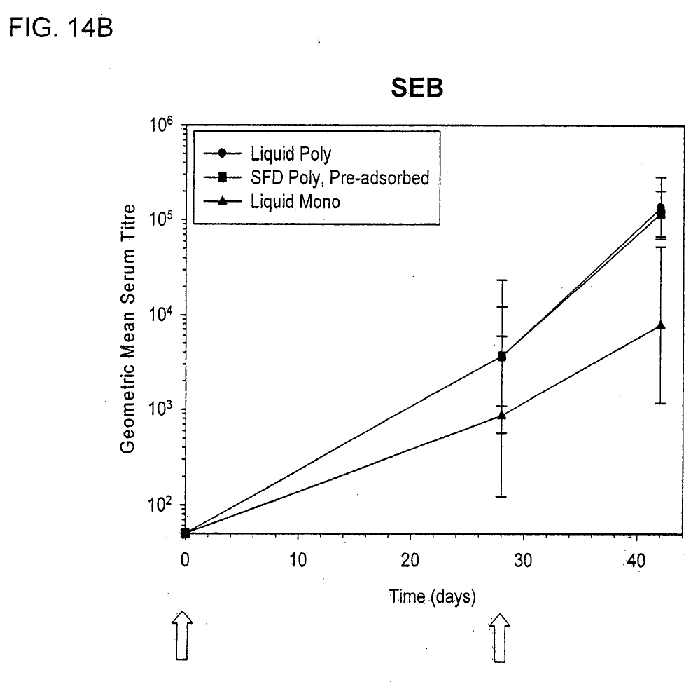

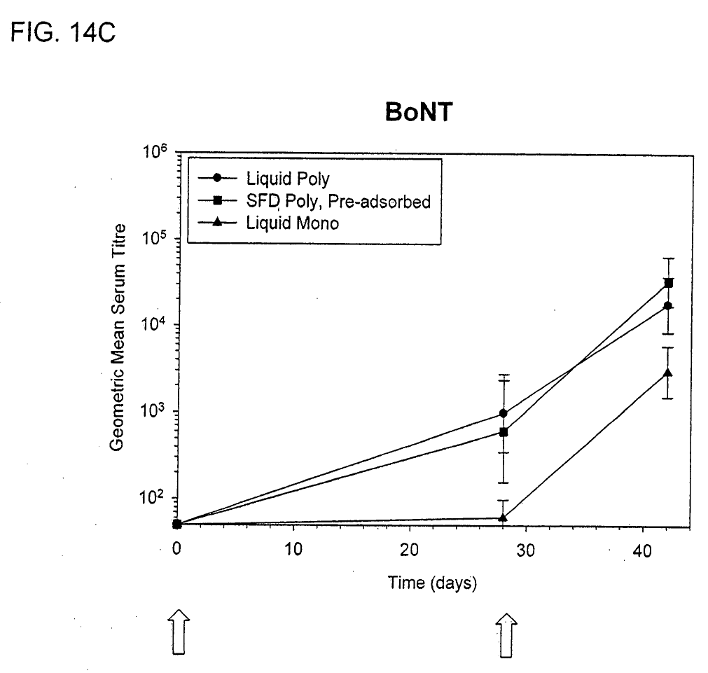

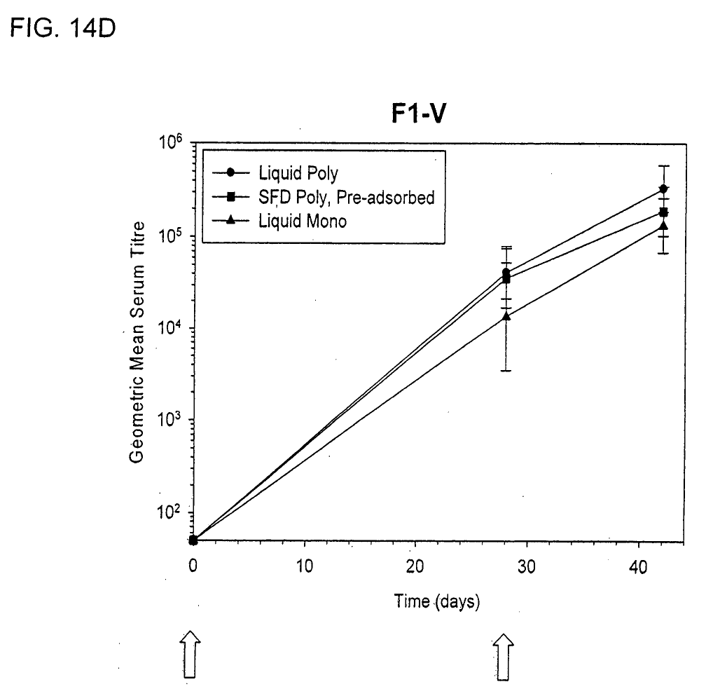

The antibody titer results obtained with pooled serum samples from mice immunized with the various vaccine formulations of Groups 1-9 on days 14, 28, and 42 are presented below in Tables 24-26, respectively. Mice immunized with liquid or reconstituted SFD polyvalent vaccine formulations containing the rPA, BoNT/A, rSEB, and F1-V antigens (i.e., test groups 1, 2, 8, and 9) generated an immune response to all four of the antigens. The antibody titers measured for each antigen in mice immunized with the polyvalent vaccine formulations were similar to those determined for each antigen in mice immunized with the monovalent control vaccine formulations (i.e., test groups 3-6), indicating that the combination of the four antigens in the polyvalent vaccine did not adversely affect the immunogenicity of each antigen. Furthermore, mice immunized with the reconstituted SFD polyvalent vaccine (i.e., test group 2) exhibited similar elevated pooled serum titers as that observed with mice immunized with the corresponding liquid polyvalent vaccine (i.e., test group 1), indicating that the SFD process did not decrease the immunogenicity of the various antigens. Similar antibody titer results were obtained when antibody titers for individual animals were analyzed. See FIG. 14.

Delivery of the liquid polyvalent vaccine formulation by IM (i.e., test group 9) or ID administration with a microneedle (i.e., test group 10) produced similar antibody titers, indicating that the vaccine is compatible with multiple administration methods. Reduction in the concentration of Alhydrogel® from 0.5% to 0.25% (i.e., test groups 8 and 9) also did not affect the immunogenicity of the polyvalent vaccine, as demonstrated by the similar antibody titers obtained with both concentrations of aluminum hydroxide (i.e., 5% and 0.25%) utilized in the polyvalent vaccines.

To confirm that the measured antibody titers were specific to the immunizing antigen, mice immunized with the monovalent vaccine controls were also screened for antibodies to antigens that were not present in the monovalent vaccine For example, rSEB, rPA, and F1-V were analyzed following immunization with the BoNT/A monovalent vaccine. Antibody titers generated by the monovalent vaccines were found to be antigen-specific at each time point, and no significant antibody levels were observed for the other antigens not included in a particular monovalent vaccine. Moreover, pooled group serum from un-immunized, naïve mice (group 7) failed to produce detectable antibody titers when screened against all four polyvalent vaccine antigens, indicating that none of the mice in this study had pre-existing immunity and further demonstrating that the titers generated in the immunized groups were specific to the immunizing antigen(s).

TABLE 24
Summary of Antibody Titers for Polyvalent Vaccine Test Groups (Day 14)

| | | | Method of | rPA/rSEB/BoNT/A/F1-V | Antigen Screened (Antibody Titers) | | | |
|---|---|---|---|---|---|---|---|---|
| Group | Formulation | Al(OH)₃ % | Delivery | Dose (μg) | F1-V | rSEB | rPA | BoNT/A |
| 1 | Liquid | 0.5 | IM | 10/20/1/10 | 6400 | 200 | 50 | <50 |
| 2 | Reconstituted | 0.5 | IM | 10/20/1/11 | 6400 | 100 | <50 | <50 |
| | SFD | | | | | | | |
| 3 | Liquid | 0.5 | IM | 10/0/0/0 | <50 | <50 | <50 | <50 |
| 4 | Liquid | 0.5 | IM | 0/20/0/0 | <50 | <50 | 50 | <50 |
| 5 | Liquid | 0.5 | IM | 0/0/1/0 | <50 | <50 | <50 | <50 |
| 6 | Liquid | 0.5 | IM | 0/0/0/10 | 3200 | <50 | <50 | <50 |
| 7 | Liquid (naïve | 0.5 | IM | 0/0/0/0 | <50 | <50 | <50 | <50 |
| | control) | | | | | | | |
| 8 | Liquid | 0.25 | IM | 10/20/1/10 | 6400 | <50 | <50 | <50 |
| 9 | Liquid | 0.25 | ID | 10/20/1/10 | 6400 | 50 | 50 | <50 |

TABLE 25
Summary of Antibody Titers for Polyvalent Vaccine Test Groups (Day 28)

| | | | Method of | rPA/rSEB/BoNT/A/F1-V | Antigen Screened (Antibody Titers) | | | |
|---|---|---|---|---|---|---|---|---|
| Group | Formulation | Al(OH)₃ % | Delivery | Dose (μg) | F1-V | rSEB | rPA | BoNT/A |
| 1 | Liquid | 0.5 | IM | 10/20/1/10 | 25600 | 6400 | 1600 | 1600 |
| 2 | Reconstituted | 0.5 | IM | 10/20/1/11 | 25600 | 6400 | 800 | 1600 |
| | SFD | | | | | | | |
| 3 | Liquid | 0.5 | IM | 10/0/0/0 | <100 | <100 | 1600 | <50 |
| 4 | Liquid | 0.5 | IM | 0/20/0/0 | <100 | 1600 | <100 | <50 |
| 5 | Liquid | 0.5 | IM | 0/0/1/0 | <100 | <100 | <100 | 1600 |
| 6 | Liquid | 0.5 | IM | 0/0/0/10 | 12800 | <100 | <100 | <50 |
| 7 | Liquid (naïve | 0.5 | IM | 0/0/0/0 | <100 | <50 | <50 | <50 |
| | control) | | | | | | | |
| 8 | Liquid | 0.25 | IM | 10/20/1/10 | 12800 | 3200 | 200 | 400 |
| 9 | Liquid | 0.25 | ID | 10/20/1/10 | 12800 | 1600 | 400 | 400 |

TABLE 26
Summary of Antibody Titers for Polyvalent Vaccine Test Groups (Day 42)

| | | | Method of | rPA/rSEB/BoNT/A/F1-V | Antigen Screened (Antibody Titers) | | | |
|---|---|---|---|---|---|---|---|---|
| Group | Formulation | Al(OH)₃ % | Delivery | Dose (μg) | F1-V | rSEB | rPA | BoNT/A |
| 1 | Liquid | 0.5 | IM | 10/20/1/10 | 102400 | 51200 | 51200 | 25600 |
| 2 | Reconstituted | 0.5 | IM | 10/20/1/11 | 102400 | 25600 | 25600 | 25600 |
| | SFD | | | | | | | |
| 3 | Liquid | 0.5 | IM | 10/0/0/0 | <200 | <200 | 12800 | <50 |
| 4 | Liquid | 0.5 | IM | 0/20/0/0 | <200 | 6400 | <200 | <50 |
| 5 | Liquid | 0.5 | IM | 0/0/1/0 | <200 | <200 | <200 | 6400 |
| 6 | Liquid | 0.5 | IM | 0/0/0/10 | 51200 | <200 | <200 | <50 |
| 7 | Liquid (naïve | 0.5 | IM | 0/0/0/0 | <100 | <100 | <100 | <50 |
| | control) | | | | | | | |
| 8 | Liquid | 0.25 | IM | 10/20/1/10 | 102400 | 25600 | 51200 | 25600 |
| 9 | Liquid | 0.25 | ID | 10/20/1/10 | 102400 | 12800 | 25600 | 25600 |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A pharmaceutical composition comprising a stable powder formulation of alum-adsorbed vaccine particles comprising at least one immunogen selected from the group consisting of a Botulinum neurotoxin (BoNT), a Bacillus anthracis antigen, a Staphylococcal enterotoxin, a Yersinia pestis antigen, and a combination thereof.

2. The pharmaceutical composition of claim 1, wherein the BoNT is BoNT/A.

3. The pharmaceutical composition of claim 1, wherein the B. anthracis antigen is B. anthracis rPA.

4. The pharmaceutical composition of claim 1, wherein the Staphylococcal enterotoxin antigen is rSEB.

5. The pharmaceutical composition of claim 1, wherein the Y. pestis immunogen is F1-V.

6. The pharmaceutical composition of claim 1, wherein the alum-adsorbed dried vaccine powder particles are reconstituted in a pharmaceutically acceptable carrier.

7. The pharmaceutical composition of claim 1, wherein the immunogen is adsorbed to an aluminum adjuvant selected from the group consisting of aluminum hydroxide, aluminum phosphate, or aluminum sulfate.

8. The pharmaceutical composition of claim 1, wherein the liquid formulation further comprises at least one excipient, wherein the at least one excipient is mannitol, trehalose, dextran, or any combination thereof.

9. The pharmaceutical composition of claim 1, wherein the composition further comprises at least one additional adjuvant.

10. A pharmaceutical composition comprising a stable powder formulation of alum-adsorbed vaccine particles comprising at least one hepatitis B immunogen, wherein the dried powder particles have an average particle size in the range of at least 80 μm to 300 μm.

11. The pharmaceutical composition of claim 10, wherein the hepatitis B antigen comprises HBsAg.

12. The pharmaceutical composition of claim 10, wherein the composition further comprises at least one immunogen selected from the group consisting of a Botulinum neurotoxin (BoNT), a Bacillus anthracis antigen, a Staphylococcal enterotoxin, a Yersinia pestis antigen, and a combination thereof.

13. The pharmaceutical composition of claim 10, wherein the alum-adsorbed dried vaccine powder particles are reconstituted in a pharmaceutically acceptable carrier.

14. The pharmaceutical composition of claim 10, wherein the immunogen is adsorbed to an aluminum adjuvant selected from the group consisting of aluminum hydroxide, aluminum phosphate, or aluminum sulfate.

15. The pharmaceutical composition of claim 10, wherein the liquid formulation further comprises at least one excipient, wherein the at least one excipient is mannitol, trehalose, dextran, or any combination thereof.

16. The pharmaceutical composition of claim 10, wherein the composition further comprises at least one additional adjuvant.

17. A method for preparing a stable powder formulation of an alum-adsorbed vaccine comprising:
a) atomizing a liquid formulation comprising at least one immunogen adsorbed onto an aluminum adjuvant to produce an atomized formulation, wherein the immunogen is selected from the group consisting of a BoNT, a B. anthracis antigen, a Staphylococcal enterotoxin antigen, a Y. pestis antigen, and a combination thereof to produce an atomized formulation;
b) freezing the atomized formulation to produce frozen particles; and
c) drying the frozen particles to produce dried powder particles.

18. The method of claim 18, wherein the liquid formulation further comprises at least one additional adjuvant.

19. A method for preparing a stable powder formulation of an alum-adsorbed vaccine comprising:
a) atomizing a liquid formulation comprising at least one hepatitis B antigen adsorbed onto an aluminum adjuvant to produce an atomized formulation, to produce an atomized formulation;
b) freezing the atomized formulation to produce frozen particles; and
c) drying the frozen particles to produce dried powder particles, wherein the dried powder particles have an average particle size of in the range of at least 80 μm to 300 μm.

20. The method of claim 19, wherein the liquid formulation further comprises at least one additional immunogen selected from the group consisting of a BoNT, a B. anthracis antigen, a Staphylococcal enterotoxin antigen, a Y. pestis antigen, and a combination thereof.

21. The method of claim 19, wherein the liquid formulation further comprises at least one additional adjuvant.

22. A method of preventing or treating in a subject anthrax, Y. pestis infection, symptoms associated with exposure to a Staphylococcal enterotoxin, or symptoms associated with exposure to a BoNT comprising administering to the subject a therapeutically effective amount of a pharmaceutical composition of claim 1.

23. A method of preventing or treating in a subject hepatitis B comprising administering to the subject a therapeutically effective amount of a pharmaceutical composition of claim 10.

24. The pharmaceutical composition of claim 1, wherein the vaccine particles have an average particle size of in the range of at least 80 μm to 300 μm.

25. The method of claim 17, wherein the dried powder particles have an average particle size of in the range of at least 80 μm to 300 μm.
